SEQUENCE LISTING (37 sequences)

SEQ ID NO: 1 - DNA, 234 bp, synthetic construct
  misc_feature 1..234: note = Recombinant Rev response element
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 2 - DNA, 126 bp, synthetic construct
  misc_feature 1..126: note = Recombinant packaging signal sequence
  source 1..126: mol_type = other DNA
ctctctcgacgcaggactcggcttgctgaagcgcgcacggcaagaggcgaggggcggcgactggtgagtacgccaaaaattttgactagcggaggctagaaggagagagatgggtgcgagagcgtc

SEQ ID NO: 3 - DNA, 181 bp, synthetic construct
  misc_feature 1..181: note = Recombinant truncated HIV-1 5 prime LTR sequence
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 4 - DNA, 234 bp, synthetic construct
  misc_feature 1..234: note = Recombinant HIV-1 self-inactivating 3 prime LTR sequence
  source 1..234: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 5 - DNA, 204 bp, synthetic construct
  misc_feature 1..204: note = Recombinant human cytomegalovirus immediate early promoter
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 6 - DNA, 118 bp, synthetic construct
  misc_feature 1..118: note = Recombinant HIV-1 central polypurine tract and central termination sequence
  source 1..118: mol_type = other DNA
ttttaaaagaaaaggggggattggggggtacagtgcaggggaaagaatagtagacataatagcaacagacatacaaactaaagaattacaaaaacaaattacaaaaattcaaaatttt

SEQ ID NO: 7 - DNA, 511 bp, synthetic construct
  misc_feature 1..511: note = Recombinant human phosphoglycerate kinase 1 promoter sequence
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 8 - DNA, 4368 bp, synthetic construct
  misc_feature 1..4368: note = Codon optimized FANCA sequence
  source 1..4368: mol_type = other DNA
atgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctga

SEQ ID NO: 9 - DNA, 380 bp, synthetic construct
  misc_feature 1..380: note = Recombinant human CMV enhancer sequence
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 10 - DNA, 122 bp, synthetic construct
  misc_feature 1..122: note = Recombinant Simian virus 40 polyA signal sequence
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 11 - DNA, 136 bp, synthetic construct
  misc_feature 1..136: note = Recombinant Simian virus 40 origin of replication sequence
  source 1..136: mol_type = other DNA
atcccgcccctaactccgcccagttccgcccattctccgccccatggctgactaattttttttatttatgcagaggccgaggccgcctcggcctctgagctattccagaagtagtgaggaggcttttttggaggcc

SEQ ID NO: 12 - DNA, 28 bp, synthetic construct
  misc_feature 1..28: note = Recombinant HIV-1 central polypurine tract and central termination sequence
  source 1..28: mol_type = other DNA
tttaaaagaaaaggggggattggggggt

SEQ ID NO: 13 - DNA, 83 bp, synthetic construct
  misc_feature 1..83: note = Recombinant dNEF signal sequence
  source 1..83: mol_type = other DNA
gaattcgagctcggtacctttaagaccaatgacttacaaggcagctgtagatcttagccactttttaaaagaaaaggggggac

SEQ ID NO: 14 - DNA, 795 bp, synthetic construct
  misc_feature 1..795: note = Recombinant KanR sequence
  source 1..795: mol_type = other DNA
atgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctga

SEQ ID NO: 15 - DNA, 137 bp, synthetic construct
  misc_feature 1..137: note = Recombinant rrnG terminator sequence
  source 1..137: mol_type = other DNA
gcattggcgcagaaaaaaatgcctgatgcgacgctgcgcgtcttatactcccacatatgccagattcagcaacggatacggcttccccaacttgcccacttccatacgtgtcctccttaccagaaatttatccttaa

SEQ ID NO: 16 - DNA, 589 bp, synthetic construct
  misc_feature 1..589: note = Recombinant high-copy-number ColE1, pMB1, pBR322, pUC origin of replication
  source 1..589: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 17 - DNA, 22 bp, synthetic construct
  misc_feature 1..22: note = Recombinant CAP binding site
  source 1..22: mol_type = other DNA
taatgtgagttagctcactcat

SEQ ID NO: 18 - DNA, 31 bp, synthetic construct
  misc_feature 1..31: note = Recombinant lac promoter sequence
  source 1..31: mol_type = other DNA
tttacactttatgcttccggctcgtatgttg

SEQ ID NO: 19 - DNA, 17 bp, synthetic construct
  misc_feature 1..17: note = Recombinant lac operator sequence
  source 1..17: mol_type = other DNA
ttgtgagcggataacaa

SEQ ID NO: 20 - DNA, 19 bp, synthetic construct
  misc_feature 1..19: note = Recombinant T3 promoter sequence
  source 1..19: mol_type = other DNA
aattaaccctcactaaagg

SEQ ID NO: 21 - DNA, 19 bp, synthetic construct
  misc_feature 1..19: note = Recombinant T7 promoter sequence
  source 1..19: mol_type = other DNA
cctatagtgagtcgtatta

SEQ ID NO: 22 - DNA, 429 bp, synthetic construct
  misc_feature 1..429: note = Recombinant f1 bacteriophage origin of replication sequence
  source 1..429: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatt

SEQ ID NO: 23 - DNA, 677 bp, synthetic construct
  misc_feature 1..677: note = Chimeric wPRE sequence
  source 1..677: mol_type = other DNA
cgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctg

SEQ ID NO: 24 - DNA, 11433 bp, synthetic construct
  misc_feature 1..11433: note = pCCL-PGK-FANCA-WPRE or pCCL-SIN-cPPT CTS-hPGK-hFANCA-WPRE
  source 1..11433: mol_type = other DNA
catgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcccccgggctgcaggaattcatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgatgagaattcgatatcaagcttatcgataccgtcgaatcccccgggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagc

SEQ ID NO: 25 - DNA, 5554 bp, synthetic construct
  misc_feature 1..5554: note = pCCL-PGK-FANCAW-82-RO
  source 1..5554: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcccccgggctgcaggaattcatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgatgagaattcgatatcaagcttatcgataccgtcgaatcccccgggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 26 - DNA, 858 bp, synthetic construct
  misc_feature 1..858: note = Recombinant Rev response element
  source 1..858: mol_type = other DNA
gatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatc

SEQ ID NO: 27 - DNA, 44 bp, synthetic construct
  misc_feature 1..44: note = Recombinant packaging signal sequence
  source 1..44: mol_type = other DNA
tgagtacgccaaaaattttgactagcggaggctagaaggagaga

SEQ ID NO: 28 - DNA, 188 bp, synthetic construct
  misc_feature 1..188: note = Recombinant truncated HIV-1 5 prime LTR sequence
  source 1..188: mol_type = other DNA
gtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgccc

SEQ ID NO: 29 - DNA, 234 bp, synthetic construct; identical to SEQ ID NO: 4
  misc_feature 1..234: note = Recombinant HIV-1 self-inactivating 3 prime LTR sequence
  source 1..234: mol_type = other DNA

SEQ ID NO: 30 - DNA, 577 bp, synthetic construct
  misc_feature 1..577: note = Recombinant human cytomegalovirus immediate early promoter sequence
  source 1..577: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 31 - DNA, 233 bp, synthetic construct
  misc_feature 1..233: note = Recombinant Rous sarcoma virus promoter sequence
  source 1..233: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 32 - DNA, 16 bp, synthetic construct
  misc_feature 1..16: note = Recombinant HIV-1 central polypurine tract and central termination sequence
  source 1..16: mol_type = other DNA
aaaagaaaagggggga

SEQ ID NO: 33 - DNA, 121 bp, synthetic construct
  misc_feature 1..121: note = Recombinant HIV-1 central polypurine tract and central termination sequence
  source 1..121: mol_type = other DNA
ttggggggtacagtgcaggggaaagaatagtagacataatagcaacagacatacaaactaaagaattacaaaaacaaattacaaaaattcaaaattttatcgatcacgagactagcctcga

SEQ ID NO: 34 - DNA, 156 bp, synthetic construct
  misc_feature 1..156: note = Recombinant Simian virus 40 origin of replication sequence
  source 1..156: mol_type = other DNA
ggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcggga

SEQ ID NO: 35 - DNA, 141 bp, synthetic construct
  misc_feature 1..141: note = Recombinant RNA-OUT sequence
  source 1..141: mol_type = other DNA
gtagaattggtaaagagagtcgtgtaaaatatcgagttcgcacatcttgttgtctgattattgatttttggcgaaaccatttgatcatatgacaagatgtgtatctaccttaacttaatgattttgataaaaatcattagg

SEQ ID NO: 36 - DNA, 4371 bp, synthetic construct
  misc_feature 1..4371: note = Codon optimized FANCA sequence
  source 1..4371: mol_type = other DNA
atgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgatga

SEQ ID NO: 37 - DNA, 1015 bp, synthetic construct
  misc_feature 1..1015: note = Recombinant high copy number ColE1, pMB1, pBR322, pUC origin of replication sequence
  source 1..1015: mol_type = other DNA
ggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

(19) United States
(12) Patent Application Publication
(10) Pub. No.: US 2026/0144823 A1
(43) Pub. Date: May 28, 2026

(54) COMPOSITIONS AND METHODS FOR STEM CELL TRANSPLANT

(71) Applicants: Spacecraft Seven, LLC, Cranbury, NJ (US); Fundación para la Investigación Biomédica del Hospital Infantil Universitario Niño Jesús, Madrid (ES)

(72) Inventors: Brian BEARD, New York, NY (US); Julian Sevilla NAVARRO, Madrid (ES); Gaurav SHAH, New York, NY (US); Kinnari PATEL, New York, NY (US); Raj PRABHAKAR, New York, NY (US)

(21) Appl. No.: 19/406,374

(22) Filed: Dec. 2, 2025

Related U.S. Application Data
(62) Division of application No. 17/045,383, now Pat. No. 12,539,316.
(60) Provisional application No. 62/656,292, filed on Apr. 11, 2018.

(51) Int. Cl. A61K 35/28; A61K 35/14; A61P 7/06; C12N 15/11

(52) U.S. Cl. CPC A61K 35/28; A61K 35/14; A61P 7/06; C12N 15/11; C12N 2310/20; C12N 2800/80

(57) ABSTRACT

The present invention provides systems and methods for manufacturing and using gene corrected stem cells for gene therapy. In particular, herein provided are methods for treating Fanconi anemia in which a subject's stem cells are selected from a biological sample obtained from the subject, genetically modified using a gene therapy vector encoding a FANC protein or a gene editing system, and administered to the subject.

### CROSS-REFERENCE TO RELATED APPLICATIONS

This application is a divisional of U.S. application Ser. No. 17/045,383, which is a National Stage application under 35 U.S.C. § 371 of International Application No. PCT/US2019/027083, filed Apr. 11, 2019, which claims the benefit under 35 U.S.C. § 119(e) of U.S. Provisional Application No. 62/656,292, filed on Apr. 11, 2018, each of which is incorporated by reference herein in its entirety.

### DESCRIPTION OF THE TEXT FILE SUBMITTED ELECTRONICALLY

The Sequence Listing XML associated with this application is provided in XML file format and is hereby incorporated by reference into the specification. The name of the XML file containing the Sequence Listing XML is ROPA_008_02US_ST26.xml. The XML file is 82,588 bytes, and created on Dec. 1, 2025, and is being submitted electronically via USPTO Patent Center.

### FIELD OF THE INVENTION

The present invention relates generally to methods of preparing populations of hematopoietic cells comprising gene-modified hematopoietic stem cells (HSC) for use in HSC transplant, including for the treatment of Fanconi anemia (FA).

### BACKGROUND OF THE INVENTION

Ex vivo mediated gene transfer into target cells is a clinically applied method for cell and gene therapy. The isolation and ex vivo genetic modification of HSC containing CD34-enriched populations provides two major benefits: reduction of gene transfer to non-target cells; and, thereby, reducing the need/amount of genetic modifiers, e.g., gene therapy vectors, which in turn reduces costs associated with clinical production of gene-modified HSCs. A model system for transplant of genetically modified HSCs is FA. Methods developed in the context of FA are also applicable to other disorders and conditions.

FA is an autosomal recessive disease (except for complementation group FA-B, which is X-linked), where the median survival of patients is around 24 years (Butturini A, et al. (1994) Blood 84:1650-1655; Kutler D I, et al. (2003) Blood 101:1249-1256). At birth, the blood count of these patients is generally normal. Macrocytosis is often the first hematological abnormality detected in these patients. This usually evolves with thrombocytopenia, anemia and pancytopenia. Bone marrow failure (BMF) is usually observed in these patients after 5-10 years, with an average age of hematologic disease onset of 7 years. About 80% of patients with FA will develop evidence of BMF in the first decade of life. Based on epidemiological studies to date, if malignant episodes do not appear before aplasia, virtually all patients with FA will develop BMF by 40 years of age (Butturini A, et al. (1994) Blood 84:1650-1655; Kutler D I, et al. (2003) Blood 101:1249-1256), this being the leading cause of mortality in these patients. Due to the complex clinical manifestations of FA, management of these patients is mainly focused on improving the following clinical manifestations: bone marrow failure (BMF), myeloid leukemia, and solid tumors.

Treatment of FA and other diseases relies on efficient engraftment of genetically modified HSCs. Accordingly, there is a need in the art for methods of preparing cell populations containing gene-modified HSCs that achieve high levels of engraftment in patients, including FA patients. The present invention addresses this need and more.

### SUMMARY OF THE INVENTION

The present invention relates generally to the fields of hematological malignancy and stem-cell transplant, and in particular, to manufacture and use of stem cell populations enriched for CD34⁺ cells for use in gene therapy, including both gene delivery and gene repair. In particular, these CD34-enriched cell populations are useful in gene therapy for the treatment of mammalian, and in particular, human diseases, disorders, and dysfunctions related to Fanconi anemia complementation group A (FANCA), group C (FANCC), or group G (FANCG) gene product dysregulation. In certain embodiments, the methods of the present disclosure are performed ex vivo and not on the human body per se.

In one embodiment, the disclosure provides a method of treating Fanconi anemia in a subject in need thereof, comprising providing to the subject a combination of: (i) a high-stringency CD34-enriched cell population prepared from a first biological sample obtained from the subject by selecting for CD34⁺ cells under high stringency conditions; and (ii) a low-stringency CD34-enriched cell population prepared from a second biological sample obtained from the subject by selecting for CD34⁺ cells under low stringency conditions, wherein one or both of the high-stringency CD34-enriched cell population and/or the low-stringency CD34-enriched cell population has been transduced with a recombinant gene therapy vector encoding a FANC polypeptide, including functional variants or fragments or naturally-occurring FANC proteins (e.g., FANCA or FANCC or FANCG), and wherein the first biological sample and the second biological sample are optionally the same biological sample, thereby treating Fanconi anemia.

In one embodiment, the method comprises treating Fanconi anemia in a subject by preparing a CD34-enriched cell population under high-stringency conditions, preparing another CD34-enriched cell population under low-stringency conditions, contacting one or both of the CD34-enriched cell populations with a recombinant gene therapy vector, and administering the two CD34-enriched cell populations, sequentially or simultaneously, to the subject, thereby treating Fanconi anemia. In particular embodiments, the cell population(s) contacted with the gene therapy vector are transduced as a result, and thus comprise a polynucleotide encoding a therapeutic nucleic acid or polypeptide for the treatment of Fanconi anemia. In an embodiment, the recombinant gene therapy vector comprises a self-inactivated lentiviral vector encoding a therapeutic FANC (e.g., FANCA or FANCC or FANCG) gene segment or protein, such as the vector described in International Patent Application No. PCT/US2017/050837. In an embodiment, the recombinant gene therapy vector is configured to repair an endogenous FANC gene (e.g., FANCA or FANCC or FANCG), such as by delivering a CRISPR/Cas system comprising a Cas protein or nucleic acid encoding a Cas protein, a gRNA or sgRNA, and a repair template comprising a FANC gene or a fragment of a FANC gene that overlaps one or more mutations to the endogenous FANC gene.

In an embodiment, the present disclosure provides, a method for treating Fanconi anemia in a subject in need thereof, comprising: preparing a high-stringency CD34-enriched cell population from a first biological sample obtained from the subject by selecting for CD34⁺ cells under high stringency conditions; preparing a low-stringency CD34-enriched cell population from a second biological sample obtained from the subject by selecting for CD34⁺ cells under low stringency conditions, contacting one or both of the high-stringency CD34-enriched cell population or the low-stringency CD34-enriched cell population with a recombinant gene therapy vector for Fanconi anemia; and administering the high-stringency CD34-enriched cell population and the low-stringency CD34-enriched cell population to the subject, wherein one or both of the high-stringency CD34-enriched cell population or the low-stringency CD34-enriched cell population is contacted with or transduced by the recombinant gene therapy vector; thereby treating Fanconi anemia.

In one aspect of the present invention, the first biological sample and the second biological sample are each independently peripheral blood or bone marrow. In one aspect, the first biological sample and the second biological sample are peripheral blood obtained after the subject has been treated with granulocyte macrophage colony-stimulating factor (G-CSF), plerixafor, or a combination of G-CSF and plerixafor.

In an embodiment, any of the methods further comprises selecting for CD34⁺ cells under high stringency conditions, which may comprise applying the first biological sample to a capture matrix that binds CD34⁺ cells, washing the capture matrix one or more times using a wash buffer, and eluting the high-stringency CD34-enriched cell population from the capture matrix using an elution buffer. In an embodiment, the applying step is performed at 5-10 mL/min, 5-15 mL/min, 15-20 mL/min, 20-25 mL/min, or 25-30 mL/min. In an embodiment, the eluting step is performed at 5-10 mL/min, 5-15 mL/min, 15-20 mL/min, 20-25 mL/min, or 25-30 mL/min. In an embodiment, the applying step is performed at 10-20 mL/min. In an embodiment, the eluting step is performed at 20 mL/min.

In an embodiment, any of the methods further comprises selecting for CD34⁺ cells under low stringency conditions, which may comprise applying the second biological sample to a capture matrix that binds CD34⁺ cells, allowing an unbound fraction of the second biological sample to flow through the capture matrix, and eluting the low-stringency CD34-enriched cell population from the capture matrix using an elution buffer. In an embodiment, the applying step is performed at 5-10 mL/min, 5-15 mL/min, 15-20 mL/min, 20-25 mL/min, or 25-30 mL/min. In an embodiment, the eluting step is performed at 5-10 mL/min, 5-15 mL/min, 15-20 mL/min, 20-25 mL/min, or 25-30 mL/min. In an embodiment, the applying step is performed at 10-20 mL/min. In an embodiment, the eluting step is performed at 20 mL/min.

In one aspect of the present invention, the high-stringency CD34-enriched cell population and/or the low-stringency CD34-enriched cell population is contacted with the recombinant gene therapy vector. In some embodiments, the percentage of CD34+ cells in the high-stringency CD34-enriched cell population is between two and four times greater than the percentage of CD34+ cells in the low-stringency CD34-enriched cell population. In an embodiment, the percentage of CD34+ cells in the high-stringency CD34-enriched cell population is greater than or about 60%, and the percentage of CD34+ cells in the low-stringency CD34-enriched cell population is less than 60% or 15-60% or between 15-30%. In an embodiment, the percentage of CD34+ cells in the high-stringency CD34-enriched cell population is greater than or about 70%, and the percentage of CD34+ cells in the low-stringency CD34-enriched cell population is 17.5-35%. In an embodiment, the percentage of CD34+ cells in the high-stringency CD34-enriched cell population is greater than or about 80%, and the percentage of CD34+ cells in the low-stringency CD34-enriched cell population is less than 40% or 20-40%. In an embodiment, the percentage of CD34+ cells in the high-stringency CD34-enriched cell population is greater than or about 90%, and the percentage of CD34+ cells in the low-stringency CD34-enriched cell population is less than 40% or 22.5-40%.

In an embodiment of the present invention, the recombinant gene therapy vector for treatment of Fanconi anemia comprises a polynucleotide sequence comprising in the following 5′ to 3′ order: (a) a eukaryotically active promoter sequence; and (b) a sequence encoding a human FANC gene or polypeptide, including functional fragments and variants thereof, wherein the sequence encoding the human FANC gene or polypeptide or functional fragment or variant thereof is operably linked to the eukaryotically active promoter sequence; and wherein the FANC gene or polypeptide is selected from FANCA, FANCC, and FANCG, e.g., native human FANCA, FANCC, and FANCG or functional fragments or variants thereof. In certain embodiments, a functional fragment or functional variant of a native FANC protein has substantially similar biological activity as the native FANC protein.

In an embodiment of the present invention, the recombinant gene therapy vector for Fanconi anemia comprises a gene editing system capable of directed repair of an endogenous FANC gene, wherein the gene editing system comprises: a Cas protein or a polynucleotide encoding a Cas protein; a gRNA; and a repair template comprising a sequence comprising the FANC gene or a fragment thereof that overlaps one or more mutations in the endogenous FANC gene; wherein the sgRNA is configured to guide the repair template to the FANC gene; and wherein the FANC gene is selected from FANCA, FANCC, and FANCG.

In an embodiment, the selection methods are performed by bead-based magnetic selection.

In an embodiment, the methods disclosed herein further comprises performing apheresis on the peripheral blood one or more times.

In one aspect, the methods disclosed herein result in progressive increase in gene-modified Fanconi Anemia cells over time.

In one aspect, the treatment methods disclosed herein inhibit the development of, halt progression of, and/or reverse progression of a hematological manifestation of Fanconi anemia in the subject, for example, without limitation, one or more of bone marrow failure, thrombocytopenia, leukopenia, pancytopenia, neutropenia, and anemia.

In one aspect, the methods disclosed herein result in recovery of one or more hematopoietic lineages that had declined in the subject prior to administration of the high-stringency CD34-enriched cell population and the low-stringency CD34-enriched cell population to the subject, for example, without limitation, one or more of lymphocytes, eosinophils, neutrophils, red blood cells, and platelets. In particular embodiments, the methods result in a slowing of or reduction in the decline of one or more hematopoietic lineages, or a stabilization in the populations of one or more hematopoietic lineages, e.g., one or more of lymphocytes, eosinophils, neutrophils, red blood cells, and platelets.

In one aspect, the method results in stabilization or recovery of one or more hematological parameters that had declined in the subject prior to administration of the high-stringency CD34-enriched cell population and the low-stringency CD34-enriched cell population to the subject, such as hemoglobin.

In an embodiment, the present disclosure provides a method for preparing genetically modified cells for the treatment of Fanconi anemia, comprising: preparing a high-stringency CD34-enriched cell population from a first biological sample obtained from a subject by selecting for CD34⁺ cells under high stringency conditions; preparing a low-stringency CD34-enriched cell population from a second biological sample obtained from a subject by selecting for CD34+ cells under low stringency conditions; and contacting one or both of the high-stringency CD34-enriched cell population or the low-stringency CD34-enriched cell population with a recombinant gene therapy vector for Fanconi anemia. In certain embodiments, the cell population(s) contacted with the gene therapy vector are transduced by the gene therapy vector, resulting in the cell populations comprising transduced cells comprising a nucleotide sequence encoding a therapeutic nucleic acid or polypeptide, e.g., a FANC polypeptide, which may be a wild type or native FANC polypeptide, or a functional fragment or variant thereof.

In one aspect, the selection of either or both low-stringency and high-stringency CD34-enriched cell populations is performed using antibodies or functional fragments thereof that specifically bind to CD34. In one aspect, selection is performed using a flow rate of 10-20 mL/min.

In an embodiment, the present invention provides a system comprising: a high-stringency CD34-enriched cell population prepared from a first biological sample by selecting for CD34⁺ cells under high stringency conditions; and a low-stringency CD34-enriched cell population prepared from a second biological sample by selecting for CD34+ cells under low stringency conditions, wherein one or both of the high-stringency CD34-enriched cell population or the low-stringency CD34-enriched cell population is contacted with or has been transduced by a recombinant gene therapy vector for Fanconi anemia. In certain embodiments, the high-stringency CD34-enriched cell population is present in a first pharmaceutical composition comprising one or more pharmaceutically acceptable carriers, diluents, or excipients, and the low-stringency CD34-enriched cell population is present in a second pharmaceutical composition comprising one or more pharmaceutically acceptable carriers, diluents, or excipients. In some embodiment, both the high-stringency CD34-enriched cell population and the low-stringency CD34-enriched cell population are present in the same pharmaceutical composition comprising one or more pharmaceutically acceptable carriers, diluents, or excipients. In certain embodiments, both the high-stringency CD34-enriched cell population and the low-stringency CD34-enriched cell population have been transduced by the gene therapy vector for treatment of Fanconi anemia. In certain embodiments, the gene therapy vector and/or the transduced cells comprise a nucleotide sequence encoding a therapeutic nucleic acid or polypeptide, e.g., a FANC polypeptide, which may be a wild type or native FANC polypeptide, or a functional fragment or variant thereof

In another embodiment, the present invention provides a pharmaceutical composition comprising: a high-stringency CD34-enriched cell population prepared from a first biological sample by selecting for CD34⁺ cells under high stringency conditions; and a low-stringency CD34-enriched cell population prepared from a second biological sample by selecting for CD34+ cells under low stringency conditions, wherein one or both of the high-stringency CD34-enriched cell population or the low-stringency CD34-enriched cell population have been transduced with a recombinant gene therapy vector. In particular embodiments, the gene therapy vector is a lentivirus. In certain embodiments, the gene therapy vector encodes a therapeutic FANC (e.g., FANCA or FANCC or FANCG) gene segment or protein, or a functional variant or fragment thereof. The pharmaceutical composition may comprise one or more pharmaceutically acceptable excipients, diluents, or carriers.

Other features and advantages of the invention will be apparent from and encompassed by the following detailed description and claims.

### BRIEF DESCRIPTION OF THE DRAWINGS

FIG. 1 shows a map of an illustrative recombinant gene therapy plasmid vector, pCCL-PGK-FANCAW-82-RO.

### DETAILED DESCRIPTION

Fanconi anemia (FA) presents several unique challenges for drug product manufacturing. For FA, as with other ex vivo gene therapy applications, the target cell population for gene transfer expresses the CD34 cell surface protein. For FA patients, when CD34+ cells are analyzed by flow cytometry, a lower proportion of BM cells are CD34+ relative to healthy individuals, 0.1-1.5% compared to 1-3%, respectively. Lower absolute CD34+ cells from drug substance manufacture from FA patient's mPB starting material was not surprising given the well-established characteristic of limited CD34+ cells in FA patients that decline with age. However, lower CD34+ yields and poor purity from FA patients were concerning, because this directly impacts the potential efficacy of the manufactured drug product.

Under standard CD34+ cell enrichment, a low percentage of CD34+ cells (relative to total cells) are bound by immunomagnetic beads; therefore, the column is loaded relatively rapidly (mL/min) and washes are stringent, because purity is the primary goal. The present inventors recognized the problem that when using mPB from FA patients, this approach provides suboptimal results with low CD34 yield.

To address the limits of standard CD34+ cell enrichment protocol for FA patients, the present inventors developed new methods for preparing CD34+ cell populations from FA patients, which result in higher yield and effective drug product. These methods involve preparing two cell populations, one selected under standard CD34+ cell enrichment conditions (“high stringency” conditions) and the other selected under “low stringency” conditions. One or both of these two cell populations are transduced with a suitable gene therapy vector and administered to the FA patient. As shown herein, the new method displays advantageous therapeutic effects. Without being bound by theory, it is believed that either the higher number of CD34+ cells or presence of other cells or other factors present in preparations of cells with lower CD34+ cell purity contribute to the surprising efficacy of compositions prepared according to the methods disclosed herein.

Accordingly, the present disclosure provides systems and methods for manufacturing and using gene-modified or gene-corrected stem cells for gene therapy. In particular, herein provided are methods for treating a disease or disorder (e.g., Fanconi anemia) in which a subject's stem cells are selected by a combination of high-stringency CD34+ selection and low-stringency CD34+ selection, transduced with a vector encoding a therapeutic agent (e.g., a FANC protein), and administered to the subject. Unexpectedly, treatment with a combination of high stringency-selected CD34+ cells and low stringency-selected CD34+ cells transduced with the therapeutic vectors, which combined have a lower CD34+ cell purity than conventional high-stringency preparations, resulted in improved therapeutic efficacy.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

In one aspect, methods and systems are provided for the manufacture of high-stringency and low-stringency CD34-enriched cell populations (e.g., by bead-based magnetic selection with antibodies or functional fragments thereof that specifically bind to CD34) from biological samples under high-stringency conditions and/or under low-stringency conditions; and for the use of these CD34-enriched cell populations in the preparation of medicaments useful in targeted gene therapy of diseases, disorders, and dysfunctions in a mammal, and in humans in particular.

In another aspect, the present invention provides a HSC transplant regimen for treating Fanconia anemia and FANC gene product-related disorders based on administering high-stringency and low-stringency CD34-enriched cell populations where either or both CD34-enriched cell populations are contacted with either a gene therapy vector (e.g., transduced with a lentiviral vector harboring the FANCA, FANCC, or FANCG gene segment), or contacted with a gene therapy vector that induces site-specific repair of FANC genes (e.g. CRISP-Cas).

In some embodiments, the biological samples are peripheral blood or bone marrow obtained from a subject, e.g., the subject to be treated. In certain embodiments, the biological samples are peripheral blood obtained after the subject has been treated with G-CSF, plerixafor, or a combination of G-CSF and plerixafor. In an embodiment, one or both of the biological samples are prepared by performing apheresis on the peripheral blood one or more times. The present disclosure refers to these methods collectively as “mobilized leukopheresis.”

In some embodiment, selecting for CD34⁺ cells under high stringency conditions comprises applying a biological sample to a capture matrix that binds CD34⁺ cells, washing the capture matrix one or more times using a wash buffer, and eluting the high-stringency CD34-enriched cell population from the capture matrix using an elution buffer. In some cases, the biological sample is re-applied to the capture matrix one or more times, such as 2, 3, 4, 5, or 6 times. In an embodiment, the applying step is performed at 5-10 mL/min, 5-15 mL/min, 15-20 mL/min, 20-25 mL/min, or 25-30 mL/min. In particular embodiments, the eluting step is performed at 5-10 mL/min, 5-15 mL/min, 15-20 mL/min, 25-25 mL/min, or 25-30 mL/min. In an embodiment, the applying step is performed at 10-20 mL/min. In an embodiment, the eluting step is performed at 20 mL/min. In some embodiments, the washing step comprises washing the capture matrix one or more times using a wash buffer. In some cases, the wash buffer comprises a volume of at least 100 mL, at least 200 mL, at least 300 mL, at least 400 mL, at least 500 mL or more. In an embodiment, the washing step is performed at 5-10 mL/min, 5-15 mL/min, 15-20 mL/min, 20-25 mL/min, or 25-30 mL/min. In an embodiment, the washing step is performed at 10-20 mL/min. In some embodiments the wash buffer is phosphate buffered saline (PBS) with, optionally, ethylenediaminetetraacetic acid (EDTA), and/or, optionally, human serum albumin at, for example, a concentration of about 2.5% w/v. In some embodiments the elution buffer is phosphate buffered saline (PBS) with, optionally, ethylenediaminetetraacetic acid (EDTA), and/or, optionally, human serum albumin at, for example, a concentration of about 2.5% w/v. In some embodiments, the wash buffer and the elution buffer are hypotonic.

In some embodiments, selecting for CD34⁺ cells under low stringency conditions comprises applying a biological sample to a capture matrix that binds CD34⁺ cells, allowing an unbound fraction of the biological sample to flow through the capture matrix, and eluting the low-stringency CD34-enriched cell population from the capture matrix using an elution buffer. In an embodiment, the applying step is performed at 1-2 mL/min, 2-3 mL/min, 1-2 mL/min, 1-2 mL/min, 5-10 mL/min, 5-15 mL/min, 15-20 mL/min. In an embodiment, the eluting step is performed at 5-10 mL/min, 5-15 mL/min, 15-20 mL/min, 25-25 mL/min, or 25-30 mL/min. In an embodiment, the applying step is performed at 10-20 mL/min. In some embodiments, the applying step is performed at an about 10%, about 20%, about 30%, about 40%, about 50%, about 60%, about 70%, about 80%, about 90% or lower flow rate than the flow rate under high stringency conditions, e.g., a flow rate at least 10% lower, at least 20% lower, at least 30% lower, at least 40% lower, at least 50% lower, at least 60% lower, at least 70% lower, at least 80% lower, or at least 90% lower than the flow rate used under high stringency conditions. In some embodiments, the washing step comprises washing the capture matrix one or more times using a wash buffer. In some cases, the wash buffer comprises a volume of 500 ml, 400 mL, 300 mL, 200 mL, 100 mL or less, e.g., less than 500 mL, less than 400 mL, less than 300 mL, less than 200 mL, or less than 100 mL. In some cases, the washing step is omitted. In an embodiment, the washing step is performed at 5-10 mL/min, 5-15 mL/min, 15-20 mL/min, 20-25 mL/min, or 25-30 mL/min. In an embodiment, the washing step is performed at 10-20 mL/min. In some embodiments the wash buffer is phosphate buffered saline (PBS) with, optionally, ethylenediaminetetraacetic acid (EDTA), and/or, optionally, human serum albumin at, for example, a concentration of about 2.5% w/v. In some embodiments the elution buffer is phosphate buffered saline (PBS) with, optionally, ethylenediaminetetraacetic acid (EDTA), and/or, optionally, human serum albumin at, for example, a concentration of about 2.5% w/v. In some embodiments, the wash buffer and the elution buffer are hypotonic.

In some embodiments, the selection is performed by bead-based magnetic selection. In an embodiment, antibodies or functional fragments thereof that specifically bind to CD34 are used for selection, e.g. bead-based magnetic selection. The selection is in some cases performed using a flow rate of 10-20 mL/min. In some cases, the flow rate is 1, 5, 10, 15, 20, 25, 30 or greater mL/min, or any value therebetween.

In embodiments, the high-stringency CD34-enriched cell population is contacted with the recombinant gene therapy vector; the low-stringency CD34-enriched cell population is contacted with the recombinant gene therapy vector; or both the high-stringency and low-stringency CD34-enriched cell populations are contacted with the recombinant gene therapy vector. In particular embodiments, the vector is a virus, a liposome, or a lipid or lipid-like nanoparticle. In some cases, the virus is a lentivirus, an adeno-associated virus, an adenovirus, or a foamy virus.

In some embodiments, the percentage of CD34+ cells in the high-stringency CD34-enriched cell population is between two and four times greater than the percentage of CD34+ cells in the low-stringency CD34-enriched cell population.

In embodiments of the methods or systems of the present invention, the recombinant gene therapy vector comprises a polynucleotide sequence comprising in the following 5′ to 3′ order: (a) a eukaryotically active promoter sequence, and (b) a sequence encoding a human FANC gene polypeptide or a functional fragment or variant thereof, and the sequence encoding the human FANC gene polypeptide or functional fragment or variant thereof is operably linked to the eukaryotically active promoter sequence. In some embodiments, the FANC gene is selected from FANCA, FANCC, and FANCG.

In an embodiment, the method inhibits the development of, halts progression of, and/or reverses progression of a hematological manifestation of a disease or disorder (e.g. Fanconi anemia) in the subject; and optionally, the hematological manifestation of Fanconi anemia is selected from one or more of bone marrow failure (BMF), thrombocytopenia, leukopenia, pancytopenia, neutropenia, and anemia. In an embodiment, the method results in progressive increase in gene-modified Franconia Anemia (or cells of a subject suffering from another disease or disorder, such as a myeloproliferative disorder or an immunodeficiency disorder) cells over time. In an embodiment, method results in recovery of one or more hematological parameters (e.g. hemoglobin) that had declined in the subject prior to administration of the high-stringency CD34-enriched cell population and the low-stringency CD34-enriched cell population to the subject.

In an embodiment, the method results in recovery of one or more hematopoietic lineages that had declined in the subject prior to administration of the high-stringency CD34-enriched cell population and the low-stringency CD34-enriched cell population to the subject; and this one or more hematopoietic lineages may comprise one or more of lymphocytes, eosinophils, neutrophils, red blood cells, and platelets. Recovery of specific cells populations is achieved in some cases. Recovery may be monitored by various methods known in the art, such as flow-assisted cell sorting, cytometry, or microscopy.

The present invention further provides compositions and systems for use in any of the embodiments of these methods. The present invention provides CD34-enriched cell populations for use in a medicament, including but not limited to CD34-enriched cell populations transduced with a gene therapy vector, e.g., a gene therapy vector comprising a polynucleotide sequence that encodes a human FANC protein, or a functional variant or fragment thereof.

As used herein, “high stringency” or “high stringency conditions” refers to a method of enriching for a cell population intended to result in substantial enrichment of cells for cells expressing a particular biological marker, e.g. CD34. For example, “high stringency” CD34 enrichment used clinically results in mean: 61.6% and median: 65.7% yield of CD34⁺ cells and mean: 88.5% and median: 95.9% relative purity (N=166) (Clin Lab. 2016 Jul. 1; 62(7):1243-1248 (PMID: 28164638)). “High stringency” refers to a process with the goal of substantial enrichment of a relatively rare cell type, CD34+, which usually comprises between 0.2-2% of the cell product in a mobilized leukopheresis or bone marrow collection. High-stringency enrichment of CD34+ cells from a mobilized leukopheresis or bone marrow collection targets final CD34+ percentages that have increased from 0.2-2% to >80%. To accomplish this, following initial application of a biological sample to a capture matrix, repeated buffer exchanges, termed herein “washes,” are carried out with the goal of removing cells weakly or non-specifically bound to the capture matrix. Generally, cells are removed from the capture matrix and reapplied for every wash cycle. Removal and reapplication can be accomplished manually by pipetting from tubes or automated using a pump and tubing system. For example, using Quad Technologies MagCloudz® coupled with Dynabeads® magnetic cell separation system, cell-magnetic particle complexes are separated in tubes on a magnetic stand and washes are done manually. Using the Miltenyi Biotec CliniMACS® System, a pre-set automated program applies the cell-magnetic particle complexes to a magnetic column in a tubing set and washes/reapplications are done using a valve pump system. In certain embodiments, selection under high stringency conditions may be performed on various instruments, including without limitation the Miltenyi Biotec MACSQuant Tyto®, Quad Technologies MagCloudz®, GE Sepax® Cell Separation System, Terumo Elutra® Cell Separation System, COBE Spectra® Cell Separator, SynGen LAB® or WASH® Systems, Fresenius-Kabi Lovo®, Miltenyi Biotec CliniMACS® System or CliniMACS Prodigy® System. Selection may be performed in a laboratory or at point-of-care. Detailed methods for preparation and enrichment of cells and cell populations, including exemplary methods for selection of CD34+ cells under high stringency conditions, are described, e.g., in Int'l Patent Pub. No. WO 2016/118780. Illustrative selection method useful for high-stringency selection are provided by U.S. Pat. No. 8,727,132.

In a high-stringency enrichment protocol, a biological sample comprising CD34+ cells is labeled with a CD34 labelling reagent, e.g. directly-conjugated immunomagnetic beads. The biological sample may be suspended in any suitable fluid, such as, without limitation, phosphate buffered saline (PBS) with, optionally, ethylenediaminetetraacetic acid (EDTA) at a buffer pH and isotonicity compatible with cell viability. In some cases, the fluid used also contain human serum albumin at a suitable concentration, such as about 2.5%. Using a magnetic activated cell sorting (MACS) technology, the biological sample, after having been labeled, is applied to a column, the column containing magnetically susceptible or ferromagnetic material. Using the MACS system, the magnetically susceptible or ferromagnetic material of the column retains the target cells without affecting the ability of non-target cells to flow through and exit the column. Such magnetically susceptible or ferromagnetic materials include iron, steel, cobalt nickel, and other ferromagnetic rare earth metals of alloys thereof. It will be appreciated by those skilled in the art that such materials may be readily magnetized and demagnetized. In some embodiments, the biological sample is recirculated over the magnetically susceptible or ferromagnetic material one or more times. Following column loading, bound cells are washed, eluted and/or re-loaded onto the column at slow speed to increase purity of the enriched fraction. Suitable wash buffers include PBS with (optionally) EDTA and (optionally) human serum albumin. Any component of the labeled biological sample which is removed during the wash steps is collected in the waste or “non-target” bag. After suitable wash steps, high-stringency enriched cells are eluted into the target cell bag.

As used herein, “low stringency” or “low-stringency conditions” refers to a method of enriching for a cell population intended to result in enrichment of cells for cells expressing a particular biological marker, e.g. CD34, in a manner that preserves a higher yield of the enriched cell population than achieved by high stringency selection at the expense of enrichment of the cells expressing the biological marker compared to other cells in the biological sample, i.e., reduced enrichment. By definition, the fold enrichment under high-stringency conditions is greater than the fold enrichment under low-stringency conditions. The fold-enrichment of cells, e.g., CD34+ cells, in the high-stringency (CD34 or other marker)-enriched cell population is, in some cases, 2, 2.25, 2.5, 2.75, 3, 3.25, 3.5, 3.75, or 4-fold the fold-enrichment of CD34+ cells in the low-stringency (CD34 or other marker)-enriched cell population. In one embodiment, the fold-enrichment of cells, e.g. CD34+ cells, in the high-stringency (CD34 or other marker)-enriched cell population is 2 to 4-fold the fold-enrichment of CD34+ cells in the low-stringency (CD34 or other marker)-enriched cell population. In certain embodiments, selection under low stringency conditions may be performed on various instruments, including without limitation the Miltenyi Biotec MACSQuant Tyto®, Quad Technologies MagCloudz®, GE Sepax® Cell Separation System, Terumo Elutra® Cell Separation System, COBE Spectra® Cell Separator, SynGen LAB® or WASH® Systems, Fresenius-Kabi Lovo®, Miltenyi Biotec CliniMACS® System or CliniMACS Prodigy® System. Selection may be performed in a laboratory or at point-of-care.

In a low-stringency enrichment protocol, a biological sample comprising CD34+ cells is labeled with a CD34 labelling reagent, e.g. directly-conjugated immunomagnetic beads. Using a magnetic activated cell sorting (MACS) technology, the biological sample, after having been labeled, is applied a column containing magnetically susceptible or ferromagnetic material at a lower flow rate than under high-stringency enrichment. As with high-stringency enrichment, the magnetically susceptible or ferromagnetic material retains the target cells without affecting the ability of non-target cells to flow through and exit the column. In some embodiments, the biological sample is recirculated over the magnetically susceptible or ferromagnetic material one or more times. Following column loading, for low-stringency enrichment, bound cells are washed at lower stringency. Bound cells are then eluted into a collection bag.

In an exemplary embodiment, low-stringency enrichment is performed by modifying the standard operating procedure of the MACS system so that a “depletion-mode” software program intended to achieve high-stringency depletion (i.e. removal of target cells) instead results in low-stringency enrichment. Operation of a MACS system in depletion mode causes target cells in the biological sample to be bound to the magnetically susceptible or ferromagnetic material in the column using slow column loading and lower stringency wash steps than operation in enrichment mode. Non-target cells are flushed by the MACS system into the wash or so-called “target” bag. The depletion-mode program then switches the output valve to direct fluid into the so-called “non-target” bag and then demagnetizes the column. Continued application of fluid over the demagnetized column results in elution of a CD34+ enriched cell population, which has been enriched under low-stringency conditions, into the so-called “non-target” bag, which using this method collects the target cells.

Those of skill in the art will recognize that this low-stringency enrichment method can be performed on various instruments, including without limitation the Miltenyi Biotec MACSQuant Tyto®, Quad Technologies MagCloudz®, GE Sepax® Cell Separation System, Terumo Elutra® Cell Separation System, COBE Spectra® Cell Separator, SynGen LAB® or WASH® Systems, Fresenius-Kabi Lovo®, Miltenyi Biotec CliniMACS® System or CliniMACS Prodigy® System. Those of skill in the art will be able, without undue experimentation, to re-program the software of such a MACs system such that the output valve directs the flow-through of the initial binding step to the waste or “non-target” bag (rather than the target bag) and directs the eluted low-stringency CD34-enriched population to the “target” bag. In effect, low-stringency enrichment is then performed in separation mode without the usual wash steps of conventional MACs programs.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors (e.g., retroviral vectors, such as lentiviral vectors), liposomes, and other gene delivery vehicles.

The term “LV” is an abbreviation for lentivirus, and may be used to refer to the virus itself or derivatives thereof. The term covers all subtypes and both naturally occurring and recombinant forms, except where required otherwise.

As used herein, the term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g., 5′ untranslated (5′ UTR) or “leader” sequences and 3′ UTR or “trailer” sequences, as well as intervening sequences (introns) between individual coding segments (exons).

As used herein, a “therapeutic gene” refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

As used herein, a transgene is a gene that is delivered to a cell by a vector.

As used herein, the term “gene product” refers to the desired expression product of a polynucleotide sequence, such as a polypeptide, peptide, protein or interfering RNA, including short interfering RNA (siRNA), miRNA or small hairpin RNA (shRNA). In certain embodiments, the gene product is a therapeutic gene product, which when expressed, confers a beneficial effect on the cell or tissue in which it is present, or on a mammal in which the gene is expressed.

As used herein, the terms “polypeptide,” “peptide,” and “protein” refer to polymers of amino acids of any length. The terms also encompass an amino acid polymer that has been modified; for example, disulfide bond formation, glycosylation, lipidation, pegylation, phosphorylation, or conjugation with a labeling component.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc. For example, a method of preparing or treating “comprising” preparing a CD34-enriched cell population is a method that may include other steps in another to preparing a CD34-enriched cell population, e.g., administering an agent to mobilize stem cells, administering an induction therapy, or co-administering a drug as a combination thereby.

The term “comprising” or “comprises” as used herein in reference to low-stringency conditions should not be construed to permit an additional high-stringency selection being applied to the same sample during the same selection step. It will be understood that performing a selection under both low-stringency conditions and under high-stringency conditions will result in a high-stringency selection, whereas low-stringency selection is expressly defined herein as excluding selection under high-stringency conditions. Preparing a low-stringency CD34-enriched cell population by selection for CD34⁺ cells under low stringency conditions may include other method steps in open-ended fashion so long as between loading the biological sample onto the column and eluting the low-stringency CD34-enriched cell population from the column, the column is not washed under high-stringency conditions.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a post-transcriptional regulatory element consists only of the promoter, polynucleotide sequence encoding the therapeutic polypeptide, and post-transcriptional regulatory element. As another example, a polypeptide “consisting of” a recited sequence contains only the recited sequence.

As used herein “bone marrow cells” or “bone marrow stem cells” refers herein to both cells obtained directly from bone marrow, such as by biopsy, and cells obtained from peripheral blood that originated in the bone marrow, e.g., following mobilization.

An “expression vector” as used herein encompasses a vector, e.g., plasmid, mini-circle, viral vector, liposome, and the like as discussed above or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g., promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer.” Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

As used herein, the terms “operatively linked” or “operably linked” refers to a juxtaposition of genetic elements, e.g., promoter, enhancer, termination signal sequence, polyadenylation sequence, etc., wherein the elements are in a relationship permitting them to operate in the expected manner. For instance, a promoter is operatively linked to a coding region if the promoter helps initiate transcription or expression of the coding sequence. There may be intervening residues between the promoter and coding region so long as this functional relationship is maintained.

As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. As another example, a promoter removed from its native coding sequence and operatively linked to a coding sequence with which it is not naturally found linked is a heterologous promoter. Thus, for example, an LV vector that includes a heterologous nucleic acid encoding a heterologous gene product is an LV vector that includes a nucleic acid not normally included in a naturally-occurring, wild-type LV, and the encoded heterologous gene product is a gene product not normally encoded by a naturally-occurring, wild-type LV.

The term “endogenous” as used herein with reference to a nucleotide molecule or gene product refers to a nucleic acid sequence, e.g., gene or genetic element, or gene product, e.g., RNA, protein, that is naturally occurring in or associated with a host virus or cell.

The term “native” as used herein refers to a nucleotide sequence, e.g., gene, or gene product, e.g., RNA, protein, that is present in a wildtype virus or cell. The term “variant” as used herein refers to a mutant or variant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the “biological activity” of an “immunoglobulin”, “antibody” or fragment or variant thereof refers to the ability to bind an antigenic determinant and thereby facilitate immunological function. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g., promoter, enhancer, kozak sequence, and the like, refers to the ability of the regulatory element or functional fragment or variant thereof to regulate, i.e., promote, enhance, or activate the translation of, respectively, the expression of the gene to which it is operably linked.

The terms “administering” or “introducing”, as used herein, refer to delivery of a cell population to a subject, e.g., by transfusing the cell population into the blood of the subject intraarterially or intravenously. The cell population may be administered in various solutions, such as saline. In some embodiments, the solution used will be isotonic to the blood of the subject and pH-buffered.

“Transformation” is typically used to refer to the process of introducing heterologous DNA into bacteria or another cell, or to cells which express an oncogene and have therefore been converted into a continuous growth mode such as tumor cells. A vector used to “transform” a cell may be a plasmid, virus or other vehicle.

Typically, a cell is referred to as “transduced”, “infected”; “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease, or slowing progression of the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease. In certain embodiments, the subject is administered the treatment following a genetic test that identified the subject as having a mutation associated with or causative for the disease, e.g., FA.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, “fragment,” as applied to a polypeptide, will ordinarily be at least 10 amino acid residues, more typically at least 20 residues, and preferably at least 3 0 (e.g., 50) residues in length, but less than the entire, intact sequence. Fragments can be generated by methods known to those skilled in the art, e.g., by enzymatic digestion of naturally occurring or recombinant protein, by recombinant DNA techniques using an expression vector that encodes a defined fragment, or by chemical synthesis. The ability of a candidate fragment to bind to a particular DNA sequence can be assessed by methods described herein. Purified fragments or antigenic fragments can be used to isolate regulatory regions or to generate new regulatory enzymes (e.g., using multiple functional fragments from different enzymes), as well as to generate antibodies, all employing standard protocols known to those skilled in the art. As used herein, “functional fragment” is meant to encompass not only those peptide fragments retaining biological activity, but also those peptide fragments that retain binding specificity to a particular nucleotide sequence.

A “functional fragment” of an anti-CD34 antibody is a fragment capable of binding to CD34 molecules on the surface of cells with sufficient affinity and specificity to permit selection or enrichment for CD34+ cells.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In some embodiments, the two sequences are the same length.

An illustrative computer program for carrying out optimal alignment, taking into consideration gap penalties is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410), the GENEWORKS suite of comparison tools, the GCG Bestfit program can be used, and BLAST 2 Sequences (see FEMS Microbiol. Lett. (1999) 174: 247-50; FEMS Microbiol. Lett. (1999) 177: 187-8). A scaled similarity score matrix may be used that assigns scores to each pairwise comparison based on chemical similarity or evolutionary distance. An example of such a matrix is the BLOSUM62 matrix—the default matrix for the BLAST suite of programs. GCG Wisconsin programs may use either the public default values or a custom symbol comparison table if supplied (see the user manual for further details).

The term “substantially identical,” in the context of two nucleic acids or polypeptides, refers to two or more sequences or subsequences that have at least 90%, at least 91%, at least 92%, at least 93%, at least 94%, at least 95%, at least 96%, at least 97%, at least 98% identity, or at least 99% identity (e.g., as determined using one of the methods set forth infra).

Unless otherwise indicated by context, a “variant” is a polypeptide or fragment thereof having one or more non-conservative or conservative amino acid substitutions relative to a second polypeptide (also referred to as a “derivative”); or a polypeptide or fragment thereof that is modified by covalent attachment of a second molecule such as, e.g., by attachment of a heterologous polypeptide, or by glycosylation, acetylation, phosphorylation, and the like. Further included within the definition of “variant” are, for example, polypeptides containing one or more analogs of an amino acid (e.g., unnatural amino acids and the like), polypeptides with unsubstituted linkages, as well as other modifications known in the art, both naturally and non-naturally occurring.

The various compositions and methods of the invention are described below. Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

The practice of the present invention employs, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, readily recognizes that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed.

Unless otherwise indicated, all terms used herein have the same meaning as they would to one skilled in the art and the practice of the present invention will employ conventional techniques of microbiology and recombinant DNA technology, which are within the knowledge of those of skill of the art.

### Methods for Preparing, Transducing, and Using CD34+-Enriched Cell Populations

In one aspect, the disclosure provides methods of preparing populations of cells enriched for CD34+ cells, including gene-modified CD34+ cells. In certain embodiments, the methods comprise preparing two CD34-enriched cell populations, one prepared using low stringency conditions, and the other prepared using high stringency conditions. As demonstrated herein, use of a combination of the low stringency CD34-enriched cell population and the high stringency CD34-enriched cell population advantageously result in unexpectedly effective reconstitution of CD34+ stem cells in a subject.

In another aspect, the disclosure provides methods of treating a subject having a disease or disorder, by providing to the subject a therapeutically effective amount of one or more cell populations enriched for CD34+ cells, including gene-modified CD34+ cells, wherein the gene-modified C34+ cells encodes a therapeutic agent effective for treating the disease or disorder. In particular embodiments, the therapeutic agent is a polypeptide or a polynucleotide, e.g., an RNA. In particular embodiments, the CD34-enriched cell populations are prepared from CD34 cells obtained from the subject being treated.

In certain embodiments of methods disclosed herein, two cell populations enriched for CD34+ cells are produced, including a high-stringency CD34-enriched cell population and a low-stringency CD34-enriched cell population. These two cell populations may be kept separate, or they may be combined to produce a mixed cell population comprising both high-stringency CD34-enriched cells and low-stringency CD34-enriched cells. In particular embodiments, either or both of the two cell populations are transduced with a gene therapy vector, e.g., an LV vector encoding FANCA or a functional fragment or variant thereof.

In particular embodiments, the cells may be from any mammalian species, e.g., rodent (e.g., mice, rats, gerbils, squirrels), rabbit, feline, canine, goat, ovine, pig, equine, bovine, primate, human. In certain embodiments, the cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

In certain embodiments, the methods comprise preparing CD34-enriched cell populations from a biological sample obtained from a subject. In one embodiment, the biological sample is a bone marrow sample. In another embodiment, the biological sample is peripheral blood.

In particular embodiments, the biological sample, e.g., peripheral blood, is obtained from the subject following mobilization of hematopoietic stem cells (HSCs). In one embodiment, HSCs and/or progenitor cells are mobilized by treating the subject with G-CSF or an analog thereof, e.g., in an amount and for a time sufficient to cause mobilization of HSCs in the patient. HSCs and progenitor cells (HSPC) in peripheral blood may be mobilized prior to collection of the biological sample. Peripheral blood HSCs and HSPC can be mobilized by any method known in the art. Peripheral blood HSCs and HSPC can be mobilized by treating the subject with any agent(s), described herein or known in the art, that increase the number of HSC and/or HSPC circulating in the peripheral blood of the subject. Reference made throughout to either HSC or HSPC are intended to encompass both HSC and HSPC unless otherwise indicated. For example, in particular embodiments, peripheral blood is mobilized by treating the subject with one or more cytokines or growth factors (e.g., G-CSF, kit ligand (KL), IL-I, IL-7, IL-8, IL-11, Flt3 ligand, SCF, thrombopoietin, or GM-CSF (such as sargramostim)). Different types of G-CSF that can be used in the methods for mobilization of peripheral blood include filgrastim and longer acting G-CSF: pegfilgrastim. In particular embodiments, peripheral blood is mobilized by treating the subject with one or more chemokines (e.g., macrophage inflammatory protein-1a (MIP1a/CCL3)), chemokine receptor ligands (e.g., chemokine receptor 2 ligands GROl3 and GR013M), chemokine receptor analogs (e.g., stromal cell derived factor-1a (SDF-1a) protein analogs such as CTCE-0021, CTCE-0214, or SDF-1a such as Met-SDF-113), or chemokine receptor antagonists (e.g., chemokine (C-X-C motif) receptor 4 (CXCR4) antagonists such as AMD3100). In particular embodiments, peripheral blood is mobilized by treating the subject with one or more anti-integrin signaling agents (e.g., function blocking anti-very late antigen 4 (VLA-4) antibody, or anti-vascular cell adhesion molecule 1 (VCAM-1)). In particular embodiments, peripheral blood is mobilized by treating the subject with one or more cytotoxic drugs such as cyclophosphamide, etoposide or paclitaxel. In particular embodiments, peripheral blood can be mobilized by administering to a subject one or more of the agents listed above for a certain period of time. For example, the subject can be treated with one or more agents (e.g., G-CSF) via injection (e.g., subcutaneous, intravenous or intraperitoneal), once daily or twice daily, for 1, 2, 3, 4, 5, 6, 7, 8, 9, 10, 11, 12, 13 or 14 days prior to collection of HSPC. In specific embodiments, HSPC are collected within 1, 2, 3, 4, 5, 6, 7, 8, 12, 14, 16, 18, 20 or 24 hours after the last dose of an agent used for mobilization of HSPC into peripheral blood. In particular embodiments, HSCs and HSPC are mobilized by treating the subject with two or more different types of agents described above or known in the art, such as a growth factor (e.g., G-CSF) and a chemokine receptor antagonist (e.g., CXCR4 receptor antagonist such as AMD3100), or a growth factor (e.g., G-CSF or KL) and an anti-integrin agent (e.g., function blocking VLA-4 antibody). In one embodiment, HSCs and/or progenitor cells are mobilized by treating the subject with G-CSF or an analog thereof. In one embodiment, the G-CSF is filgrastim. In one embodiment, HSCs and/or progenitor cells are mobilized by treating the subject with plerixafor. In a certain embodiment, HSCs and/or progenitor cells are mobilized using a combination of filgrastim and plerixafor, by filgrastim alone, or by plerixafor alone. In particular embodiments, different types of mobilizing agents are administered concurrently or sequentially. For additional information regarding methods of mobilization of peripheral blood see, e.g., Craddock et al., 1997, Blood 90(12):4779-4788; Jin et al., 2008, Journal of Translational Medicine 6:39; Pelus, 2008, Curr. Opin. Hematol. 15(4):285-292; Papayannopoulou et al., 1998, Blood 91(7):2231-2239; Tricot et al., 2008, Haematologica 93(11):1739-1742; and Weaver et al., 2001, Bone Marrow Transplantation 27(2):S23-S29).

In certain embodiments, peripheral blood is obtained through a syringe or catheter inserted into a subject's vein. For example, the peripheral blood can be collected using an apheresis machine. Blood flows from the vein through the catheter into an apheresis machine, which separates the white blood cells, including HSPC from the rest of the blood and then returns the remainder of the blood to the subject's body. Apheresis can be performed for several days (e.g., 1 to 5 days) until enough HSPC have been collected.

In certain embodiments, bone marrow is obtained from the posterior iliac crest of the subject by needle aspiration (see, e.g., Koda et al., 1984, J. Clin Invest. 73:1377-1384).

In certain embodiments, a hematocrit level of the biological sample may be determined. The hematocrit level may be determined by centrifuging the sample within a treatment chamber to separate RBCs of a sample into a layer such that the packed cell volume may be determined. It should be appreciated that the sample may be combined with an anticoagulant in order to assist with determining the hematocrit level and that such an anticoagulant may be added to the treatment chamber prior to or during centrifugation. Alternatively, the hematocrit level may be determined by measuring optical properties of the sample. For example, a spectrometer may be used to analyze the sample. It should be appreciated that any type of known spectroscopic methods of determining hematocrit level may be used such as, for example, Raman spectroscopy and/or light scattering techniques.

In certain embodiments, the biological sample is depleted of erythrocytes, e.g., before preparing the one or more cell populations enriched for CD34+ cells from the biological sample. In some embodiments, the cells remaining after depletion techniques are washed. In another embodiment, non-specific IgG is added to the washed cells. In some embodiments, the non-specific IgG is flebogamma.

Two populations of cells enriched for CD34+ cells may be produced by selecting a population of cells for CD34+ cells under high-stringency conditions and selecting another population of cells for CD34+ cells under low stringency conditions, thereby producing two cell populations, one a high-stringency CD34-enriched cell population and the other a low-stringency CD34-enriched cell population. Methods used to select for CD34+ cells may be positive selection, negative selection, or a combination thereof. In certain embodiments, the biological sample obtained from the subject is divided into two samples, where one sample is used to prepare the high-stringency CD34-enriched cell population, and the other sample is used to prepare the low-stringency CD34-enriched cell population.

In other embodiments, the biological sample obtained from the subject is first subjected to a low-stringency CD34+ selection to prepare a low-stringency CD34-enriched cell population, and then a portion of the low-stringency CD34-enriched population is subjected to a high-stringency CD34+ selection to prepare a high-stringency CD34-enriched cell population. Selection may be applied sequentially, e.g., a selection for CD34-enriched cells under low stringency conditions may be applied first followed by selection from the resulting population of further CD34-enriched cells under high stringency conditions.

In other cases, selection for CD34-enriched cells under high stringency conditions may be applied first followed by selection from the residual population of CD34-enriched cells under low stringency conditions. In some cases, the cell populations may be split such that a low stringency or a high stringency selection is applied to a fraction of the cells subjected to high stringency or low stringency selection previously. In some cases, one biological sample is split into two or more samples before selection of CD34-enriched cells under low or high stringency conditions.

In some cases, two or more biological samples are mixed together before selection, including, e.g. mobilized bone marrow samples acquired at different times, such as 1, 2, 3, 4 or more days apart, or 1, 2, or 3 weeks apart, or 1, 2, or 3 months apart, or years apart, inclusive of other time increments. In cases, biological samples from different subjects are mixed, such as, e.g., an autologous biological sample and a sample from an allogenic donor. In every case, high-stringency or low-stringency selection preceding or following mixing or splitting biological samples or enriched cell populations is contemplated, in all possible permutations. In certain embodiments, the method comprises preparing a high-stringency CD34-enriched cell population from a first biological sample obtained from the subject by selecting for CD34⁺ cells under high stringency conditions; and preparing a low-stringency CD34-enriched cell population from a second biological sample obtained from the subject by selecting for CD34⁺ cells under low stringency conditions.

In some embodiments, the percentage of CD34+ cells in the high-stringency CD34-enriched cell population is between 1.5-fold, 2-fold, 2.5-fold, 3-fold, 3.5-fold, 4-fold greater, or in some instances 5-fold, 6-fold, 7-fold, or 8-fold greater than the percentage of CD34+ cells in the low-stringency CD34-enriched cell population.

Various yields and purity of CD34+ cells may be achieved by the methods disclosed. In some cases, selection of CD34+ cells under high stringency conditions results in high-stringency CD34-enriched cell populations with yields of CD34+ cells compared to input biological sample of at least about 10%, 20%, 30%, 40%, 50%, 60%, 70%, 80% or greater, or any value in between; and/or in purity compared to the total number of cells in the enriched sample of at least about 20%, 30%, 40%, 50%, 60%, 70%, 80%, 90%, 95% or greater, or any value in between. In some cases, selection of CD34+ cells under high stringency conditions results in high-stringency CD34-enriched cell populations with yields of CD34+ cells compared to input biological sample of about 10% to 20%, 20% to 30%, or 30% to 40%, or any value in between; and/or in purity compared to the total number of cells in the enriched sample of at least about 20%, 30%, 40%, 50%, or any value in between. In particular embodiments, selection of CD34+ cells under high stringency conditions results in high-stringency CD34-enriched cell populations with yields of CD34+ cells compared to input biological sample of at least about 20%; and/or in purity compared to the total number of cells in the enriched sample of at least about 20% or at least about >30%.

In some cases, selection of CD34+ cells under low stringency conditions results in low-stringency CD34-enriched cell populations with yields of CD34+ cells compared to input biological sample of at least about 30%, 40%, 50%, 60%, 70%, 80%, 90%, 95% or greater, or any value in between; and in purity compared to the total number of cells in the enriched sample of at least about 5%, 10%, 15%, 25%, 40%, 50% or greater, or any value in between. In some cases, selection of CD34+ cells under low stringency conditions results in low-stringency CD34-enriched cell populations with yields of CD34+ cells compared to input biological sample of greater than 30%, about 30% to 40%, 40% to 50%, or 50% to 60%, or any value in between; and in purity compared to the total number of cells in the enriched sample of at least about 3%, 5%, 8%, 10%, or any value in between. In particular embodiments, selection of CD34+ cells under low stringency conditions results in high-stringency CD34-enriched cell populations with yields of CD34+ cells compared to input biological sample of greater than 35%; and/or in purity compared to the total number of cells in the enriched sample of less than 30%, less than 25%, less than 20%, less than 10%, or less than 5%. In particular embodiments, selection of CD34+ cells under low stringency conditions results in low-stringency CD34-enriched cell populations with purity compared to the total number of cells in the enriched sample of 1-30%, 1-20%, 1-10%, 10-30%, 10-20%, or 20-30%. In some embodiments, the low-stringency CD34-enriched cell populations further comprise at least neutrophils and B cells.

In particular embodiments, high-stringency and low-stringency selection are both performed by positive selection, e.g., using an agent that binds CD34+ cells. In particular embodiments, the agent is in solution, whereas in other embodiments, the agent is affixed to a solid surface. In certain embodiments, the agent is bound to the surface of a magnetic bead. In some embodiments, the agent is an anti-CD34 antibody or functional fragment thereof.

In certain embodiments, selection under high stringency conditions and/or low stringency conditions may be performed on various instruments, including without limitation the Miltenyi Biotec MACSQuant Tyto®, Quad Technologies MagCloudz®, GE Sepax® Cell Separation System, Terumo Elutra® Cell Separation System, COBE Spectra® Cell Separator, SynGen LAB® or WASH® Systems, Fresenius-Kabi Lovo®, Miltenyi Biotec CliniMACS® System or CliniMACS Prodigy® System. Selection may be performed in a laboratory or at point-of-care. Detailed methods for preparation and enrichment of cells and cell populations, including exemplary methods for selection of CD34+ cells under high stringency conditions, are described, e.g., in Int'l Patent Pub. No. WO 2016/118790. Illustrative selection method useful for high-stringency selection are provided by U.S. Pat. No. 8,727,132.

In an embodiment, the instrument is a closed system device that includes material inputs (e.g., sample, buffers, gas) at least one treatment chamber with centrifugation and cell incubation capabilities, a closed tubing set, a pump, and a target cell selector. Controlling software enables the device to isolate, genetically-modify, and formulate target cells ex vivo, in particular embodiments, directly from a subject sample. In particular embodiments, the selection process can be completed within 30 hours, within 25 hours or within 20 hours with minimal to no user input. In particular embodiments, the entire process is completed within 72 hours or within 64 hours. In particular embodiments, minimal user input means that between sample input into the device and recovery of genetically-modified cells formulated for administration to a subject, the user interacts with the device no more than 20, 15, 10, or 5 times and/or interacts with the device for no more than 12 hours, 10 hours, 8 hours, 5 hours, 4 hours, or 3 hours. Exemplary interactions from a user can include one or more of: connecting a sterile tubing set; verifying maintenance of a closed, sterile system; determining that a stage should be repeated (e.g., sedimentation); verifying successful completion of a stage; allowing a new stage to begin following a process quality check; providing reagents for device input; and determining and/or calculating volumes for addition or removal. Interactions can be timed by, following sample receipt, the amount of time the user is preparing for or actually interacting with the device.

In certain embodiments, one or both of the high-stringency CD34-enriched cell population and/or low-stringency CD34-enriched cell population is prepared using a CliniMACS® instrument, such as the CliniMACS®^{plus} Instrument, which is a software controlled instrument that processes the HSC, apherisis. The CliniMACS®^{plus} Instrument, and related equipment and reagents, are commercially available from Miltenyi Biotec GmbH, Bergisch Gladbach, Germany). Equipment or reagents used include the CliniMACS® CD34 antigen/reagent, which is a solution containing an antibody conjugate consisting of a murine IgG, monoclonal antibody directed against the Class II epitope of the human CD34 antigen, which is chemically conjugated to dextran beads having an iron oxide/hydroxide core, which is used for selection or enrichment of CD34+ cells; CliniMACS® Tubing, which are single-use, sterile, disposable tubing set with two cell separation columns for processing up to 0.6×10⁹ CD34+ cells out of a total cell number not exceeding 60×10⁹ white blood cells (standard-scale) or up to 1.2×10⁹ CD34+ cells out of a total cell number not exceeding 120×10⁹ white blood cells (large-scale); and CliniMACS® PBS/EDTA Buffer, which is a sterile, isotonic phosphate-buffered, 1 mM EDTA, saline solution, used as an external wash and transport fluid for the in vitro processing of HSC, apheresis. Washes can be performed using CliniMACS Buffer comprising PBS and 2.5% human serum albumin. Procedures for using the CliniMACS® system are provided in the CliniMACS User Manuals available at the Miltenyi website, including, e.g., the CliniMACS® User Manual for the CliniMACS® CD34 Reagent System obtained on Apr. 8, 2019 at www.miltenyibiotec.com/_Resources/Persistent/2c28c84939f4ce793b29c9f2e897c0bb1a334c47/User%20Manual%20for%20the%20CliniMACS%20CD34%20Reagent%20System.pdf. In one embodiment, the high stringency CD34-enriched cell population is prepared by labelling the cells using the CD34 reagent, and then the CD34+ cells are selected using a standard CliniMACS® CD34 enrichment program. In one embodiment, the low stringency CD34-enriched cell population is prepared using a modified version of a CliniMACS® depletion program. According to this embodiment, the cells are labelled using the CD34 reagent, but a modified depletion program is run. After loading of the labelled cells with the magnet ON, the Cell Collection bag is removed and the collected cells not used for transplant. Instead, the magnet is turned OFF and elution buffer is applied to the instrument, resulting in elution of the CD34+ cells, which are collected. In certain embodiments, as compared to selection mode, under depletion mode, the instrument is loaded more slowly (mL/min), and the washes are lower stringency.

In certain embodiments, one or both of the high-stringency CD34-enriched cell population and/or low-stringency CD34-enriched cell population is transduced with a vector, e.g., a gene therapy vector that encodes a therapeutic agent, e.g., by contacting either or both of the high-stringency CD34-enriched cell population and/or the low-stringency CD34-enriched cell population with the vector. In certain embodiments, the cells are contacted with the vector for about 30 minutes, about 1 hour, about 1.5 hours, about 2 hours, about 2.5 hours, about 3 hours, about 3.5 hours, about 4 hours, about 5 hours, about 6 hours, about 7 hours, about 8 hours, about 12 hours, about 16 hours, about 18 hours, about 20 hours, about 24 hours, about 36 hours, about 48 hours, about 60 hours. In some embodiments, the cells are transduced for less than 60 hours, less than 48 hours, less than 36 hours, or less than 24 hours. In certain embodiments, the cells may be contacted with the vector one or more times, e.g. one time, twice, three times, or more than three times, and the cells allowed to incubate with the agent(s) for some amount of time following each contacting event e.g. 16-24 hours, after which time the media is replaced with fresh media and the cells are cultured further. Contacting the cells with the vector may occur in any culture media and under any culture conditions that promote the survival of the cells. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

In certain embodiments, cells are contacted with an effective amount of gene therapy vector sufficient to achieve detectable levels of the encoded therapeutic agent. In particular embodiments, the therapeutic agent is a polypeptide or polynucleotide useful or effective for the treatment of a disease or disorder. In particular embodiments, the therapeutic agent is a polypeptide that is deregulated or mutated in the cells obtained from the subject, e.g., a FANC polypeptide for the treatment of FA. The effective amount may be readily determined empirically, e.g. by detecting the presence or levels of the therapeutic agent, by detecting an effect on the viability or function of the cells, etc. Typically, expression of the therapeutic agent will be enhanced 2-fold or more, for example 3-fold, 4-fold, or 5-fold or more, in some instances 10-fold, 20-fold or 50-fold or more, e.g. 100-fold in transduced cells as compared to non-transduced cells.

Any convenient gene therapy vector that finds use delivering polynucleotide sequences to mammalian cells is encompassed by the gene therapy vectors of the present disclosure. For example, the vector may comprise single or double stranded nucleic acid, e.g. single stranded or double stranded DNA. For example, the gene therapy vector may be DNA, e.g., a naked DNA, e.g., a plasmid, a minicircle, etc. The vector may comprise single-stranded or double-stranded RNA, including modified forms of RNA. In another example, the gene therapy vector may be an RNA, e.g., an mRNA or modified mRNA.

In particular embodiments, the gene therapy vector may be a viral vector derived from a virus, e.g., an adenovirus, an adeno-associated virus, a lentivirus (LV), a herpes virus, an alphavirus or a retrovirus, e.g., Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) or Rous Sarcoma Virus (RSV). While embodiments encompassing the use of LV are described in greater detail below, it is expected that the ordinarily skilled artisan will appreciate that similar knowledge and skill in the art can be brought to bear on non-LV gene therapy vectors as well. In some embodiments, the gene therapy vector is a self-limiting LV.

In particular, certain methods disclosed herein relate to transducing one or both populations of stem cells or progenitor cells, e.g., hematopoietic stem cells (HSCs) or hematopoietic progenitor cells (also referred to herein as “hematopoietic progenitors”) with a gene therapy vector encoding and/or expressing a therapeutic polypeptide, e.g., FANCA, where one population is prepared by selection under high-stringency conditions and the other population is prepared by selection under low-stringency conditions In one embodiment, the cell populations are enriched for CD34+ cells. In one embodiment, the HSCs or hematopoietic progenitors are from a subject with diminished or no protein activity from one or more FANCA encoded proteins. In one embodiment, the subject has FA-A. In one embodiment, the endogenous FANCA gene of the HSCs is deleted and/or mutated.

In one embodiment, transducing a cell with a gene therapy vector results in the integration into the cell genome of an expression cassette comprising a promoter operably linked to a polynucleotide sequence encoding a therapeutic agent within the gene therapy vector. In some embodiments, transducing a cell with a gene therapy vector results in the expression of the therapeutic agent, e.g., a biologically active FANCA protein.

For example, a biologically active FANCA protein forms part of the FA core complex. In certain embodiments, a FANCA gene is delivered via a viral vector. In one embodiment, a FANCA gene is delivered via a lentiviral vector. In certain embodiments, the lentiviral vector is PGK-FANCA.WPRE*LV. It is contemplated that after transduction of bone marrow (BM) cells or stem cells or progenitor cells from FA-A patients with a FANCA lentiviral vector (LV), the therapeutic vector is integrated in the genome of the cells. Once integrated, the therapeutic protein (e.g., human FANCA protein) is expressed by the cells. Transduced FA cells are genetically corrected, and thus able to activate the FA pathway by the mono-ubiquitination of FANCD2 and FANCI. These proteins will be then able to migrate to areas of DNA damage, and in cooperation with other DNA repair proteins, will promote the repair of the DNA in these cells, as occurs in healthy cells.

As discussed herein, the subject methods and compositions find use in expressing a transgene, e.g., FANCA, in cells of an animal. For example, the subject compositions may be used in research, e.g., to determine the effect that the gene has on cell viability and/or function. As another example, the subject compositions may be used in medicine, e.g., to treat a disorder such as FA.

Accordingly, the present disclosure provides methods of treating a disease or disorder in a subject in need thereof, comprising providing or administering to the subject a combination of a high-stringency CD34-enriched cell population and a low-stringency CD34-enriched cell population prepared as described herein, wherein one or both of the high-stringency CD34-enriched cell population or the low-stringency CD34-enriched cell population is transduced with a recombinant gene therapy vector comprising a polynucleotide sequence encoding a therapeutic agent or comprising an expression cassette that expresses the therapeutic agent, wherein the therapeutic agent is effective in treating the disease or disorder. In particular embodiments, the disease is FA, and the therapeutic agent is a Fanconi anemia complementation group A (FANCA) polypeptide, or a functional variant or fragment thereof.

In particular embodiments, the method comprises: (a) preparing a high-stringency CD34-enriched cell population from a first biological sample obtained from the subject by selecting for CD34⁺ cells under high stringency conditions; (b) preparing a low-stringency CD34-enriched cell population from a second biological sample obtained from the subject by selecting for CD34⁺ cells under low stringency conditions, (c) transducing one or both of the high-stringency CD34-enriched cell population or the low-stringency CD34-enriched cell population with the recombinant gene therapy vector comprising the polynucleotide sequence encoding the therapeutic agent; and (d) providing or administering the high-stringency CD34-enriched cell population and the low-stringency CD34-enriched cell population to the subject, wherein one or both of the high-stringency CD34-enriched cell population or the low-stringency CD34-enriched cell population is transduced with the recombinant gene therapy vector; wherein the therapeutic agent is effective in treating the disease or disorder. In particular embodiments, the disease is FA, and the therapeutic agent is a Fanconi anemia complementation group A (FANCA) polypeptide, or a functional variant or fragment thereof. In certain embodiments, all of the cells obtained from each of the two cell populations is administered to the patient. The cells doses of each of the two cell populations may be similar or different, e.g., with more total cells and more total CD34+ cells coming from the low stringency population. In certain embodiments, the cells originally obtained from the patient are divided into two approximately equal populations, one population being processed under high stringency CD34+ selection conditions and one under low stringency CD34+ selection condition, which may result in ˜2-fold more CD34+ cells and 5-10-fold more total cells resulting from the low stringency selection process.

In particular embodiments of any of the methods of treatment described herein, the first biological sample and the second biological sample are each independently peripheral blood or bone marrow. In certain embodiments, the first biological sample and the second biological sample are peripheral blood obtained after the subject has been treated with G-CSF, plerixafor, or a combination of G-CSF and plerixafor.

In particular embodiments of the methods of treatment described herein, the selecting for CD34⁺ cells under high stringency conditions comprises loading the first biological sample onto a column that binds CD34⁺ cells, washing the loaded column one or more times using a wash buffer, and eluting the high-stringency CD34-enriched cell population from the column using an elution buffer. In an embodiment, the applying step is performed at 5-10 mL/min, 5-15 mL/min, 15-20 mL/min, 20-25 mL/min, or 25-30 mL/min. In an embodiment, the eluting step is performed at 5-10 mL/min, 5-15 mL/min, 15-20 mL/min, 25-25 mL/min, or 25-30 mL/min. In an embodiment, the applying step is performed at 10-20 mL/min. In an embodiment, the eluting step is performed at 20 mL/min.

In particular embodiments of the methods of treatment described herein, the selecting for CD34⁺ cells under low stringency conditions comprises of loading the second biological sample onto a column that binds CD34⁺ cells, allowing the second biological sample to flow through the column, and eluting the low-stringency CD34-enriched cell population from the column using an elution buffer. In an embodiment, the applying step is performed at 5-10 mL/min, 5-15 mL/min, 15-20 mL/min, 20-25 mL/min, or 25-30 mL/min. In an embodiment, the eluting step is performed at 5-10 mL/min, 5-15 mL/min, 15-20 mL/min, 25-25 mL/min, or 25-30 mL/min. In an embodiment, the applying step is performed at 10-20 mL/min. In an embodiment, the eluting step is performed at 20 mL/min.

In particular embodiments of the methods of treatment described herein, the high-stringency CD34-enriched cell population is transduced and/or the low-stringency CD34-enriched cell population is transduced.

In some embodiments, the subject methods result in a therapeutic benefit, e.g., preventing the development of a disorder, halting the progression of a disorder, reversing the progression of a disorder, etc. For example, in one embodiment, the disorder is FA. In another embodiment, the disease or disorder is BMF. In one embodiment, the disorder is thrombocytopenia. In another embodiment, the disorder is leukopenia. In one embodiment, the disorder is pancytopenia. In one embodiment, the disorder is neutropenia. In another embodiment, the disorder is anemia. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

As described in further detail in the Examples, clinical data with bone marrow cells mobilized into peripheral blood biological samples and administered according to methods of the present invention have demonstrated, unexpectedly, more rapid, in vivo selection engraftment kinetics relative to the other FA patients transplanted by prior art methods. In particular embodiments, methods and composition of the present disclosure are used to treat FA.

Accordingly, the present invention provides methods for treatment of FA, or one or more of the hematological manifestations of FA. In one embodiment, the hematological manifestation of FA is selected from one or more of bone marrow failure (BMF), thrombocytopenia, leukopenia, pancytopenia, neutropenia, and anemia. In a particular embodiment, the hematological manifestation is BMF, which appears in pediatric ages in most FA patients. In one embodiment, the hematological manifestation is thrombocytopenia. In another embodiment, the hematological manifestation is leukopenia. In one embodiment, the hematological manifestation is pancytopenia. In one embodiment, the hematological manifestation is neutropenia. In another embodiment, the hematological manifestation is anemia. In one embodiment, the hematological manifestation is a combination of two or more of BMF, thrombocytopenia, leukopenia, pancytopenia, neutropenia, and anemia.

It is also an object of the present invention to repair endogenous FANC genes (e.g. FANCA, FANCC, and/or FANCG) using a CRISPR/Cas gene editing system or the like. Cas9-mediated repair of genes related to Fanconi anemia has been demonstrated in vitro, e.g. Obsorn et al. Fanconi anemia gene editing by the CRISPR/Cas9 system. Hum Gene Ther. 2015 February; 26(2):114-26. The present invention provides methods for treating Fanconi anemia and method of preparing genetically modified cells that result in improved in vivo efficiency of repair and improved reconstitution of bone marrow. In some cases, the gene editing system repairs a single nucleotide polymorphism, a deletion, an insertion, an indel, or another genetic defect. The repair is in some cases directed to a coding region of a gene, an intron, or an upstream or downstream region, such as a gene-regulatory region of the genome proximal or distant from a gene associated with a disease or disorder.

In one embodiment of the methods of the present invention, the disclosure provides a method for treating Fanconi anemia in a subject in need thereof that comprises preparing a high-stringency CD34-enriched cell population from a first biological sample obtained from the subject by selecting for CD34⁺ cells under high stringency conditions; preparing a low-stringency CD34-enriched cell population from a second biological sample obtained from the subject by selecting for CD34⁺ cells under low stringency conditions, contacting one or both of the high-stringency CD34-enriched cell population or the low-stringency CD34-enriched cell population with a recombinant gene therapy vector for Fanconi anemia; and administering the high-stringency CD34-enriched cell population and the low-stringency CD34-enriched cell population to the subject, wherein one or both of the high-stringency CD34-enriched cell population or the low-stringency CD34-enriched cell population is contacted with the recombinant gene therapy vector; and wherein the gene therapy vector comprises a gene editing system capable of directed repair of an endogenous gene, thereby treating the Fanconi anemia. In some cases, the gene editing system is a CRISPR-Cas system (e.g. CRISPR-Cas9).

In some embodiments, the gene editing system comprises a nucleic acid encoding a Cas, Cas9, or spCas gene product (such as an mRNA, cDNA, plasmid, or the like) operatively linked to a promoter. In some embodiments, the gene editing system comprises a nucleic acid encoding a Cas, Cas9, or spCas protein. In some cases, the gene therapy vector is liposome or lipid nanoparticle or other lipid-based or lipid-like delivery system. In some cases, the gene therapy vector is a virus, such as a lentivirus, adenovirus, or adeno-associated virus. In some embodiments, the gene editing system comprises a guide RNA, e.g. a single-guide RNA (sgRNA), and optionally, a repair template. The repair template and the guide RNA are in some cases distinct molecules or in some cases the same molecule. The repair template and the guide RNA can be covalently or non-covalently linked. The guide RNA is in some cases pre-loaded into a Cas, Cas9, or spCas protein. The components of the gene editing system are in some cases delivered in a single gene therapy vector. In some cases, the gene editing system is delivered in distinct gene therapy vectors. Multiple gene therapy vectors can be used. For example, and without limitation, multiple genetic lesions can be repaired with the same or with different gene therapy vectors. In some cases, two or more FANC genes are repaired simultaneously. In some cases, one gene therapy vector is contacted with the high-stringency CD34-enriched cell populations and another gene therapy vector is contacted with the low-stringency CD34-enriched cell populations. In some cases, the two gene therapy vectors are the same. In some cases, one gene therapy vector comprises a transgene for a disorder associated with a given gene (e.g. FANCA) and the other gene therapy vector comprises a gene editing system.

In some cases, the gene therapy vector provides a transgene for, or repairs, a gene other than a gene associated with a disease or disorder. For example, without limitation, the gene therapy vector may up or down regulate immune effector genes, may alter cell surface markers, may provide alternate MHC molecules or may encode immunoglobulin genes. It is particularly contemplated that in some cases the gene therapy vector or vectors provide for use of allogenic or unmatch donor transplant, such as by altering immune markers (e.g HLA or MHC genes) or causing expression of immune effector genes.

In some embodiments, the disclosure provides a method of treating a disease or disorder in a subject in need thereof, comprising providing to the subject a combination of a high-stringency CD34-enriched cell population and a low-stringency CD34-enriched cell population, both prepared from one or more biological sample obtained from a subject wherein the CD34 cells obtained from the subject comprise one or more gene mutation associated with or causative of the disease or disorder, and wherein one or both of the high-stringency CD34-enriched cell population or the low-stringency CD34-enriched cell population is subjected to gene editing to repair the one or more gene mutation prior to providing the cell populations to the subject. In certain embodiments, the gene editing is performed by contacting the one or both CD34-enriched cell population with a Cas (e.g. Cas9 or spCas) protein; a guide RNA (e.g., single guide RNA, sgRNA); and a repair template. In particular embodiments, the disease is FA, and the gene mutation that is repaired is a mutation in a Fanconi anemia complementation group A (FANCA) gene.

In certain embodiments, gene therapy vector disclosed herein comprise a polynucleotide comprising a promoter operably linked to a sequence encoding a therapeutic agent, i.e., a coding sequence. As used here, the term “operably linked” means that the promoter is capable of driving expression of the sequence encoding the therapeutic agent.

In some embodiments, the polynucleotide comprises one or more enhancers. Enhancers are nucleic acid elements known in the art to enhance transcription, and can be located anywhere in association with the gene they regulate, e.g. upstream, downstream, within an intron, etc. Any enhancer element can be used in the polynucleotide cassettes and gene therapy vectors of the present disclosure, so long as it enhances expression of the gene when used in combination with the promoter.

The coding sequence to be expressed in the cells can be any polynucleotide sequence, e.g. gene or cDNA that encodes a gene product, e.g. a polypeptide or RNA-based therapeutic (siRNA, antisense, ribozyme, shRNA, etc.). The coding sequence may be heterologous to the promoter sequence to which it is operably linked, i.e. not naturally operably associated with it. Alternatively, the coding sequence may be endogenous to the promoter sequence to which it is operably linked, i.e. is associated in nature with that promoter. The gene product may act intrinsically in the mammalian cell, or it may act extrinsically, e.g., it may be secreted. For example, when the transgene is a therapeutic gene, the coding sequence may be any gene that encodes a desired gene product or functional fragment or variant thereof that can be used as a therapeutic for treating a disease or disorder. In various embodiments, the transgene encodes human FANCA.

In one embodiment of the invention, the transgene coding sequence is modified, or “codon optimized” to enhance expression by replacing infrequently represented codons with more frequently represented codons. The coding sequence is the portion of the mRNA sequence that encodes the amino acids for translation. During translation, each of 61 trinucleotide codons are translated to one of 20 amino acids, leading to a degeneracy, or redundancy, in the genetic code. However, different cell types, and different animal species, utilize tRNAs (each bearing an anticodon) coding for the same amino acids at different frequencies. When a gene sequence contains codons that are infrequently represented by the corresponding tRNA, the ribosome translation machinery may slow, impeding efficient translation. Expression can be improved via “codon optimization” for a particular species, where the coding sequence is altered to encode the same protein sequence, but utilizing codons that are highly represented, and/or utilized by highly expressed human proteins (Cid-Arregui et al., 2003; J. Virol. 77: 4928). In one aspect of the present invention, the coding sequence of the transgene is modified to replace codons infrequently expressed in mammal or in primates with codons frequently expressed in primates. For example, in some embodiments, the coding sequence encoded by the transgene encodes a polypeptide having at least 85% sequence identity to a polypeptide encoded by a sequence disclosed above or herein, for example at least 90% sequence identity, e.g. at least 95% sequence identity, at least 98% identity, at least 99% identity, wherein at least one codon of the coding sequence has a higher tRNA frequency in humans than the corresponding codon in the sequence disclosed above or herein.

In an additional embodiment of the invention, the transgene coding sequence is modified to enhance expression by termination or removal of open reading frames (ORFs) that do not encode the desired transgene. An open reading frame (ORF) is the nucleic acid sequence that follows a start codon and does not contain a stop codon. ORFs may be in the forward or reverse orientation, and may be “in frame” or “out of frame” compared with the gene of interest. Such open reading frames have the potential to be expressed in an expression cassette alongside the gene of interest, and could lead to undesired adverse effects. In one aspect of the present invention, the coding sequence of the transgene has been modified to remove open reading frames by further altering codon usage. This may be done by eliminating start codons (ATG) and introducing stop codons (TAG, TAA, or TGA) in reverse orientation or out-of-frame ORFs, while preserving the amino acid sequence and maintaining highly utilized codons in the gene of interest (i.e., avoiding codons with frequency<20%). In the present disclosure, the transgene coding sequence may be optimized by either of codon optimization and removal of non-transgene ORFs or using both techniques. As will be apparent to one of ordinary skill in the art, it is preferable to remove or minimize non-transgene ORFs after codon optimization in order to remove ORFs introduced during codon optimization.

The present disclosure includes plasmids comprising an expression cassette or transfer cassette described herein. In particular embodiments, the plasmid is pCCL-PGK-FANCA-WPRE* (SEQ ID NO: 24) and/or the plasmid comprises the expression cassette from pCCL-PGK-FANCA-WPRE (SEQ ID NO: 25).

In certain embodiments, a gene therapy vector or gene transfer cassette therein comprises one or more additional elements, e.g., one or more elements selected from the following: 5′ LTR, 3′LTR, cPPT, CTS, RRE, enhancer sequences, and packaging signals. In some embodiments, the gene therapy vector or gene transfer cassette is any vector or cassette disclosed in Int'l Pat. Pub. No. WO 2018/049273 A1, the disclosure of which is incorporated herein in its entirety for all purposes. Polynucleotide sequences encoding a Fanconi anemia complementation group (FANC) polypeptide include those disclosed in U.S. Pat. No. 5,952,190, the disclosure of which is incorporated herein in its entirety for all purposes.

The RRE sequence improves the efficiency of gene transfer. In particular embodiments of any of the expression cassettes and gene therapy vectors described herein, the RRE sequence comprises or consists of any of the following sequences, or sequences having at least 80%, at least 85%, at least 90%, at least 95%, at least 98%, or at least 99% identity to the following sequences (all displayed in the 5′ to 3′ orientation):

(SEQ ID NO: 1)
AGGAGCTTTGTTCCTTGGGTTCTTGGGAGCAGCAGGAAGCACTATGGGCG
CAGCGTCAATGACGCTGACGGTACAGGCCAGACAATTATTGTCTGGTATA
GTGCAGCAGCAGAACAATTTGCTGAGGGCTATTGAGGCGCAACAGCATCT
GTTGCACTCACAGTCTGGGGCATCAAGCAGCTCCAGGCAAGAATCCTGGC
TGTGGAAAGATACCTAAAGGATCAACAGCTCCT;
(SEQ ID NO: 26)
GATCTTCAGACCTGGAGGAGGAGATATGAGGGACAATTGGAGAAGTGAAT
TATATAAATATAAAGTAGTAAAAATTGAACCATTAGGAGTAGCACCCACC
AAGGCAAAGAGAAGAGTGGTGCAGAGAGAAAAAAGAGCAGTGGGAATAGG
AGCTTTGTTCCTTGGGTTCTTGGGAGCAGCAGGAAGCACTATGGGCGCAG
CGTCAATGACGCTGACGGTACAGGCCAGACAATTATTGTCTGGTATAGTG
CAGCAGCAGAACAATTTGCTGAGGGCTATTGAGGCGCAACAGCATCTGTT
GCAACTCACAGTCTGGGGCATCAAGCAGCTCCAGGCAAGAATCCTGGCTG
TGGAAAGATACCTAAAGGATCAACAGCTCCTGGGGATTTGGGGTTGCTCT
GGAAAACTCATTTGCACCACTGCTGTGCCTTGGAATGCTAGTTGGAGTAA
TAAATCTCTGGAACAGATTTGGAATCACACGACCTGGATGGAGTGGGACA
GAGAAATTAACAATTACACAAGCTTAATACACTCCTTAATTGAAGAATCG
CAAAACCAGCAAGAAAAGAATGAACAAGAATTATTGGAATTAGATAAATG
GGCAAGTTTGTGGAATTGGTTTAACATAACAAATTGGCTGTGGTATATAA
AATTATTCATAATGATAGTAGGAGGCTTGGTAGGTTTAAGAATAGTTTTT
GCTGTACTTTCTATAGTGAATAGAGTTAGGCAGGGATATTCACCATTATC
GTTTCAGACCCACCTCCCAACCCCGAGGGGACCCGACAGGCCCGAAGGAA
TAGAAGAAGAAGGTGGAGAGAGAGACAGAGACAGATCCATTCGATTAGTG
AACGGATC;

or
a sequence comprising or consisting of nucleotides 2649-2882 of SEQ ID NO:24.

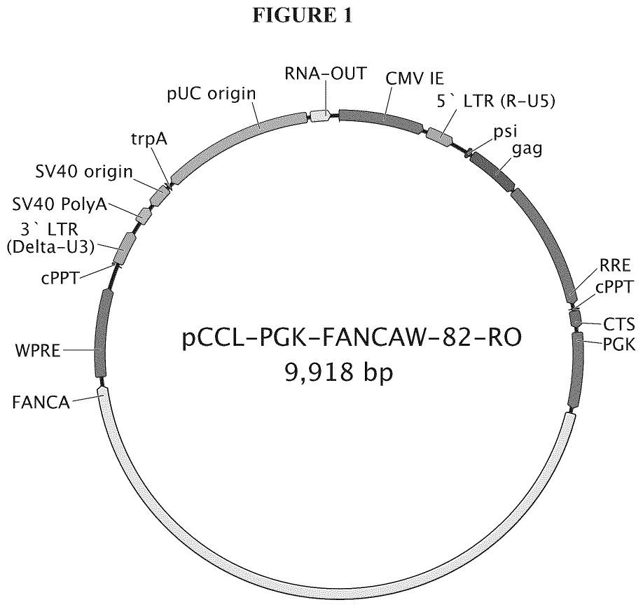

The retroviral leader region contains the packaging signal (Ψ), which is involved in packaging the retroviral genome into the viral capsid. LV vectors were thought to require approximately 300 bp of the Gag gene in this region. Currently, this Gag sequence has been reduced to just 40 bp (FIG. 1). In particular embodiments of any of the expression cassettes and gene therapy vectors described herein, the ψ sequence is an HIV-1 ψ sequence or the T sequence comprises or consists of any of the following sequences, or sequences having at least 80%, at least 85%, at least 90%, at least 95%, at least 98%, or at least 99% identity to the following sequences:

(SEQ ID NO: 2)
CTCTCTCGACGCAGGACTCGGCTTGCTGAAGCGCGCACGGCAAGAGGCGA
GGGGCGGCGACTGGTGAGTACGCCAAAAATTTTGACTAGCGGAGGCTAGA
AGGAGAGAGATGGGTGCGAGAGCGTC;
(SEQ ID NO: 27)
TGAGTACGCCAAAAATTTTGACTAGCGGAGGCTAGAAGGAGAGA;

or
a sequence comprising or consisting of polynucleotides 2031-2156 of SEQ ID NO:24.

In particular embodiments of any of the expression cassettes and gene therapy vectors described herein, the truncated HIV-1 5′ LTR comprises or consists of any of the following sequences, or sequences having at least 80%, at least 85%, at least 90%, at least 95%, at least 98%, or at least 99% identity to any of the following sequences:

(SEQ ID NO: 3)
GGGTCTCTCTGGTTAGACCAGATCTGAGCCTGGGAGCTCTCTGGCTAAC
TAGGGAACCCACTGCTTAAGCCTCAATAAAGCTTGCCTTGAGTGCTTCA
AGTAGTGTGTGCCCGTCTGTTGTGTGACTCTGGTAACTAGAGATCCCTC
AGACCCTTTTAGTCAGTGTGGAAAATCTCTAGCA;
(SEQ ID NO: 28)
GTCTCTCTGGTTAGACCAGATCTGAGCCTGGGAGCTCTCTGGCTAACTA
GGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAG
ACCCTTTTAGTCAGTGTGGAAAATCTCTAGCAGTGGCGCCC;

or
a sequence comprising or consisting of polynucleotides 1804-1984 of SEQ ID NO:24.

In particular embodiments of any of the expression cassettes and gene therapy vectors described herein, the HIV-1 self-inactivating 3′ LTR comprises or consists of any of the following sequences, or sequences having at least 80%, at least 85%, at least 90%, at least 95%, at least 98%, or at least 99% identity to the following sequences:

(SEQ ID NO: 4)
TGGAAGGGCTAATTCACTCCCAACGAAGACAAGATCTGCTTTTTGCTTG
TACTGGGTCTCTCTGGTTAGACCAGATCTGAGCCTGGGAGCTCTCTGGC
TAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATC
CCTCAGACCCTTTTAGTCAGTGTGGAAAATCTCTAGCA;
(SEQ ID NO: 29)
TGGAAGGGCTAATTCACTCCCAACGAAGACAAGATCTGCTTTTTGCTTG
TACTGGGTCTCTCTGGTTAGACCAGATCTGAGCCTGGGAGCTCTCTGGC
TAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATC
CCTCAGACCCTTTTAGTCAGTGTGGAAAATCTCTAGCA;

or
a sequence comprising or consisting of polynucleotides 9262-9495 of SEQ ID NO:24.

In particular embodiments of any of the expression cassettes and gene therapy vectors described herein, the human cytomegalovirus (CMV) immediate early promoter comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 5)
GTGATGCGGTTTTGGCAGTACATCAATGGGCGTGGATAGCGGTTTGACT
CACGGGGATTTCCAAGTCTCCACCCCATTGACGTCAATGGGAGTTTGTT
TTGGCACCAAAATCAACGGGACTTTCCAAAATGTCGTAACAACTCCGCC
CCATTGACGCAAATGGGCGGTAGGCGTGTACGGTGGGAGGTCTATATAA
GCAGAGCT;
(SEQ ID NO: 30)
ACATTGATTATTGACTAGTTATTAATAGTAATCAATTACGGGGTCATTA
GTTCATAGCCCATATATGGAGTTCCGCGTTACATAACTTACGGTAAATG
GCCCGCCTGGCTGACCGCCCAACGACCCCCGCCCATTGACGTCAATAAT
GACGTATGTTCCCATAGTAACGCCAATAGGGACTTTCCATTGACGTCAA
TGGGTGGAGTATTTACGGTAAACTGCCCACTTGGCAGTACATCAAGTGT
ATCATATGCCAAGTACGCCCCCTATTGACGTCAATGACGGTAAATGGCC
CGCCTGGCATTATGCCCAGTACATGACCTTATGGGACTTTCCTACTTGG
CAGTACATCTACGTATTAGTCATCGCTATTACCATGGTGATGCGGTTTT
GGCAGTACATCAATGGGCGTGGATAGCGGTTTGACTCACGGGGATTTCC
AAGTCTCCACCCCATTGACGTCAATGGGAGTTTGTTTTGGCACCAAAAT
CAACGGGACTTTCCAAAATGTCGTAACAACTCCGCCCCATTGACGCAAA
TGGGCGGTAGGCGTGTACGGTGGGAGGTCTATATAAGC

or
a sequence comprising or consisting of polynucleotides 1586-1789 of SEQ ID NO:24.

In particular embodiments of any of the expression cassettes and gene therapy vectors described herein, the Rous sarcoma virus (RSV) promoter comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 31)
TTAATGTAGTCTTATGCAATACTCTTGTAGTCTTGCAACATGGTAACGA
TGAGTTAGCAACATGCCTTACAAGGAGAGAAAAAGCACCGTGCATGCCG
ATTGGTGGAAGTAAGGTGGTACGATCGTGCCTTATTAGGAAGGCAACAG
ACGGGTCTGACATGGATTGGACGAACCACTGAATTGCCGCATTGCAGAG
ATATTGTATTTAAGTGCCTAGCTCGATACAATAAACG.

The cPPT, which facilitates nuclear translocation of the pre-integration complexes, together with the CTS involved in the separation of reverse transcriptase, has been seen to improve viral titer (Zennou, et al. 2000; Follenzi et al. 2000). In particular embodiments of any of the expression cassettes and gene therapy vectors described herein, the central polypurine tract and central termination sequence of HIV-1 (cPPT/CTS) comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 6)
TTTTAAAAGAAAAGGGGGGATTGGGGGGTACAGTGCAGGGGAAAGAATA
GTAGACATAATAGCAACAGACATACAAACTAAAGAATTACAAAAACAAA
TTACAAAAATTCAAAATTTT;
(SEQ ID NO: 12)
TTTAAAAGAAAAGGGGGGATTGGGGGGT;
(SEQ ID NO: 32)
AAAAGAAAAGGGGGGA;
(SEQ ID NO: 33)
TTGGGGGGTACAGTGCAGGGGAAAGAATAGTAGACATAATAGCAACAGA
CATACAAACTAAAGAATTACAAAAACAAATTACAAAAATTCAAAATTTT
ATCGATCACGAGACTAGCCTCGA

or
a sequence comprising or consisting of nucleotides 3378-3495 of SEQ ID NO:24.

In particular embodiments of any of the expression cassettes and gene therapy vectors described herein, the human phosphoglycerate kinase 1 (hPGK) promoter comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 7)
GGGGTTGGGGTTGCGCCTTTTCCAAGGCAGCCCTGGGTTTGCGCAGGGA
CGCGGCTGCTCTGGGCGTGGTTCCGGGAAACGCAGCGGCGCCGACCCTG
GGTCTCGCACATTCTTCACGTCCGTTCGCAGCGTCACCCGGATCTTCGC
CGCTACCCTTGTGGGCCCCCCGGCGACGCTTCCTGCTCCGCCCCTAAGT
CGGGAAGGTTCCTTGCGGTTCGCGGCGTGCCGGACGTGACAAACGGAAG
CCGCACGTCTCACTAGTACCCTCGCAGACGGACAGCGCCAGGGAGCAAT
GGCAGCGCGCCGACCGCGATGGGCTGTGGCCAATAGCGGCTGCTCAGCA
GGGCGCGCCGAGAGCAGCGGCCGGGAAGGGGCGGTGCGGGAGGCGGGGT
GTGGGGCGGTAGTGTGGGCCCTGTTCCTGCCCGCGCGGTGTTCCGCATT
CTGCAAGCCTCCGGAGCGCACGTCGGCAGTCGGCTCCCTCGTTGACCGA
ATCACCGACCTCTCTCCCCAG;

or
a sequence comprising or consisting of nucleotides 3541-4051 of SEQ ID NO:24 or nucleotides 1-511 of SEQ ID NO:25.

In certain embodiments, expression cassettes and gene therapy vectors disclosed herein further comprises one or more additional elements, e.g., a CMV promoter and/or enhancer, an SV40 polyA sequence, an origin of replication, e.g., an SV40 on sequence, or any of the elements disclosed herein.

In particular embodiments of expression cassettes and gene therapy vectors described herein, the human CMV enhancer comprises or consists of the following sequence, a functional fragment thereof, or a sequence having at least 80%, at least 85%, at least 90%, at least 95%, at least 98%, or at least 99% identity to the following sequence:

(SEQ ID NO: 9)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATG.

In particular embodiments of any of the expression cassettes and gene therapy vectors described herein described herein, the simian virus 40 (SV40) poly(A) signal comprises or consists of the following sequence, a functional fragment thereof, or a sequence having at least 80%, at least 85%, at least 90%, at least 95%, at least 98%, or at least 99% identity to the following sequence:

(SEQ ID NO: 10)
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA.

In particular embodiments of any of the expression cassettes and gene therapy vectors described herein described herein, the SV40 origin of replication comprises or consists of the following sequence, a functional fragment thereof, or a sequence having at least 80%, at least 85%, at least 90%, at least 95%, at least 98%, or at least 99% identity to the following sequence:

(SEQ ID NO: 11)
ATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGC
TATTCCAGAAGTAGTGAGGAGGCTTTTTTGGAGGCC;
(SEQ ID NO: 34)
GGCCTCCAAAAAAGCCTCCTCACTACTTCTGGAATAGCTCAGAGGCCGAG
GCGGCCTCGGCCTCTGCATAAATAAAAAAAATTAGTCAGCCATGGGGCGG
AGAATGGGCGGAACTGGGCGGAGTTAGGGGCGGGATGGGCGGAGTTAGGG
GCGGGA.

In some embodiments of any of the expression cassettes and gene therapy vectors described herein described herein, the dNEF signal present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 13)
GAATTCGAGCTCGGTACCTTTAAGACCAATGACTTACAAGGCAGCTGTAG
ATCTTAGCCACTTTTTAAAAGAAAAGGGGGGAC.

In particular embodiments of any of the expression cassettes and gene therapy vectors described herein described herein, the KanR sequence present in any of the expression cassettes or gene therapy vectors described herein comprises or consists of the following sequence:

(SEQ ID NO: 14)
ATGATTGAACAAGATGGATTGCACGCAGGTTCTCCGGCGGCTTGGGTGGA
GAGGCTATTCGGCTATGACTGGGCACAACAGACAATCGGCTGCTCTGATG
CCGCCGTGTTCCGGCTGTCAGCGCAGGGGCGTCCGGTTCTTTTTGTCAAG
ACCGACCTGTCCGGTGCCCTGAATGAACTGCAAGACGAGGCAGCGCGGCT
ATCGTGGCTGGCGACGACGGGCGTTCCTTGCGCGGCTGTGCTCGACGTTG
TCACTGAAGCGGGAAGGGACTGGCTGCTATTGGGCGAAGTGCCGGGGCAG
GATCTCCTGTCATCTCACCTTGCTCCTGCCGAGAAAGTATCCATCATGGC
TGATGCAATGCGGCGGCTGCATACGCTTGATCCGGCTACCTGCCCATTCG
ACCACCAAGCGAAACATCGCATCGAGCGAGCACGTACTCGGATGGAAGCC
GGTCTTGTCGATCAGGATGATCTGGACGAAGAGCATCAGGGGCTCGCGCC
AGCCGAACTGTTCGCCAGGCTCAAGGCGTCTATGCCCGACGGCGAGGATC
TCGTCGTGACCCACGGCGATGCCTGCTTGCCGAATATCATGGTGGAAAAT
GGCCGCTTTTCTGGATTCATCGACTGTGGCCGTCTGGGTGTGGCGGACCG
CTATCAGGACATAGCGTTGGCTACCCGTGATATTGCTGAAGAGCTTGGCG
GCGAATGGGCTGACCGCTTCCTTGTGCTTTACGGTATCGCCGCGCCCGAT
TCGCAGCGCATCGCCTTCTATCGCCTTCTTGACGAGTTCTTCTGA.

In particular embodiments of any of the expression cassettes and gene therapy vectors described herein described herein, the RNA-OUT sequence present in any of the expression cassettes or gene therapy vectors described herein comprises or consists of the following sequence:

(SEQ ID NO: 35)
GTAGAATTGGTAAAGAGAGTCGTGTAAAATATCGAGTTCGCACATCTTGT
TGTCTGATTATTGATTTTTGGCGAAACCATTTGATCATATGACAAGATGT
GTATCTACCTTAACTTAATGATTTTGATAAAAATCATTAGG.

In some embodiments of any of the expression cassettes and gene therapy vectors described herein described herein, the rrnG terminator (transcription terminator from the E. coli ribosomal RNA rrnG operon (Albrechtsen et al., 1991) present in any of the expression cassettes or gene therapy vectors described herein comprises or consists of the following sequence:

(SEQ ID NO: 15)
GCATTGGCGCAGAAAAAAATGCCTGATGCGACGCTGCGCGTCTTATACTC
CCACATATGCCAGATTCAGCAACGGATACGGCTTCCCCAACTTGCCCACT
TCCATACGTGTCCTCCTTACCAGAAATTTATCCTTAA

In some embodiments of any of the expression cassettes and gene therapy vectors described herein, the ori (high-copy-number ColE1/pMIB1/pBR322/pUC origin of replication) present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 16)
TTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTT
TTTCCGAAGGTAACTGGCTTCAGCAGAGCGCAGATACCAAATACTGTTCT
TCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGC
GATAAGTCGTGTCTTACCGGGTTGGACTCAAGACGATAGTTACCGGATAA
GGCGCAGCGGTCGGGCTGAACGGGGGGTTCGTGCACACAGCCCAGCTTGG
AGCGAACGACCTACACCGAACTGAGATACCTACAGCGTGAGCTATGAGAA
AGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGA
TTTTTGTGATGCTCGTCAGGGGGGCGGAGCCTATGGAAA
(SEQ ID NO: 37)
GGAGTCAGGCAACTATGGATGAACGAAATAGACAGATCGCTGAGATAGGT
GCCTCACTGATTAAGCATTGGTAACTGTCAGACCAAGTTTACTCATATAT
ACTTTAGATTGATTTAAAACTTCATTTTTAATTTAAAAGGATCTAGGTGA
AGATCCTTTTTGATAATCTCATGACCAAAATCCCTTAACGTGAGTTTTCG
TTCCACTGAGCGTCAGACCCCGTAGAAAAGATCAAAGGATCTTCTTGAGA
TCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCG
AAGGTAACTGGCTTCAGCAGAGCGCAGATACCAAATACTGTTCTTCTAGT
GTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAG
TCGTGTCTTACCGGGTTGGACTCAAGACGATAGTTACCGGATAAGGCGCA
GCGGTCGGGCTGAACGGGGGGTTCGTGCACACAGCCCAGCTTGGAGCGAA
CGACCTACACCGAACTGAGATACCTACAGCGTGAGCTATGAGAAAGCGCC
ACGCTTCCCGAAGGGAGAAAGGCGGACAGGTATCCGGTAAGCGGCAGGGT
CGGAACAGGAGAGCGCACGAGGGAGCTTCCAGGGGGAAACGCCTGGTATC
TTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTC
CTGCGTTATCCCCTGATTCTGTGGATAACCGTATTACCGCCTTTGAGTGA
GCTGATACCGCTCGCCGCAGCCGAACGACCGAGCGCAGCGAGTCAGTGAG
CGAGGAAGCGGAAGA;

or
a sequence comprising or consisting of nucleotides 9731-9871 of SEQ ID NO: 24.

In some embodiments of any of the expression cassettes and gene therapy vectors described herein described herein, the CAP binding site present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 17)
TAATGTGAGTTAGCTCACTCAT.

In some embodiments of any of the expression cassettes and gene therapy vectors described herein described herein, the E. coli lac promoter present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 18)
TTTACACTTTATGCTTCCGGCTCGTATGTTG.

In some embodiments of any of the expression cassettes and gene therapy vectors described herein described herein, the lac operator present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 19)
TTGTGAGCGGATAACAA.

In some embodiments of any of the expression cassettes and gene therapy vectors described herein described herein, the T3 promoter (promoter for bacteriophage T3 RNA polymerase) present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 20)
AATTAACCCTCACTAAAGG.

In some embodiments of any of the expression cassettes and gene therapy vectors described herein described herein, the T7 promoter (promoter for bacteriophage T7 RNA polymerase) present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 21)
CCTATAGTGAGTCGTATTA.

In some embodiments of any of the expression cassettes and gene therapy vectors described herein described herein, the f1 on (f1 bacteriophage origin of replication) present in any of the expression cassettes or gene therapy vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 22)
ACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTG
GACTCTTGTTCCAAACTGGAACAACACTCAACCCTATCTCGGTCTATTCT
TTTGATTTATAAGGGATTTTGCCGATTTCGGCCTATTGGTTAAAAAATGA
GCTGATTTAACAAAAATTTAACGCGAATT.

As discussed herein, the expression cassettes and gene therapy vectors of the present invention may comprise an RNA export signal. Exemplary RNA export sequences include but are not limited to WPRE. The WPRE significantly increases transgene expression in target cells, by increasing RNA stability in a transgene, promoter and vector-independent manner (Zuffrey et al, 1999). However, it can express a truncated 60-amino acid protein derived from the WHV X gene involved in liver cancer (Kingsman et al, 2005). Therefore, most pre-clinical protocols and clinical trials include a mutated version of the WPRE element (Zanta-Boussif et al, 2009). On the other hand, the use of two SV40-USE elements in SIN-LV vectors has been seen to be more efficient than the WPRE sequence in suppressing transcriptional read through (Schambach et al, 2007). More precisely, the WPRE disclosed herein is a chimeric wPRE that carries 589 nucleotides from the modified WPRE performed by Axel Schambach (nucleotides 1-589) (WO 2008136670 A2) and 88 from a former WPRE (nucleotide 590-677) (Zuffrey et al, 1999). Data disclosed herein shows this chimeric WPRE works better than the former wPRE. The chimeric wPRE sequence comprises the following sequence, a functional fragment thereof, or a sequence having at least 80%, at least 85%, at least 90%, at least 95%, at least 98%, or at least 99% identity to the following sequence:

(SEQ ID NO: 23)
CGAGCATCTTACCGCCATTTATTCCCATATTTGTTCTGTTTTTCTTGATT
TGGGTATACATTTAAATGTTAATAAAACAAAATGGTGGGGCAATCATTTA
CATTTTTAGGGATATGTAATTACTAGTTCAGGTGTATTGCCACAAGACAA
ACATGTTAAGAAACTTTCCCGTTATTTACGCTCTGTTCCTGTTAATCAAC
CTCTGGATTACAAAATTTGTGAAAGATTGACTGATATTCTTAACTATGTT
GCTCCTTTTACGCTGTGTGGATATGCTGCTTTAATGCCTCTGTATCATGC
TATTGCTTCCCGTACGGCTTTCGTTTTCTCCTCCTTGTATAAATCCTGGT
TGCTGTCTCTTTATGAGGAGTTGTGGCCCGTTGTCCGTCAACGTGGCGTG
GTGTGCTCTGTGTTTGCTGACGCAACCCCCACTGGCTGGGGCATTGCCAC
CACCTGTCAACTCCTTTCTGGGACTTTCGCTTTCCCCCTCCCGATCGCCA
CGGCAGAACTCATCGCCGCCTGCCTTGCCCGCTGCTGGACAGGGGCTAGG
TTGCTGGGCACTGATAATTCCGTGGTGTTGTCGGGGAAGGGCCTGCTGCC
GGCTCTGCGGCCTCTTCCGCGTCTTCGCCTTCGCCCTCAGACGAGTCGGA
TCTCCCTTTGGGCCGCCTCCCCGCCTG.

In particular embodiments, the mutated WPRE sequence comprises or consists of WPRE*, which corresponds to nucleotides 8502-9178 of SEQ ID NO: 24, or has at least 80%, at least 85%, at least 90%, at least 95%, at least 98%, or at least 99% identity to this region of SEQ ID NO: 24.

Other combinations of elements both as disclosed herein or as known in the art will be readily appreciated by the ordinarily skilled artisan.

Additionally, as will be recognized by one of ordinary skill in the art, the expression cassettes and gene therapy vectors may optionally contain other elements including, but not limited to restriction sites to facilitate cloning and regulatory elements for a particular gene therapy vector.

In some aspects of the present invention, the subject polynucleotide cassettes are used to deliver a gene to cells, e.g. to determine the effect that the gene has on cell viability and/or function, to treat a cell disorder, etc. In various embodiments, delivery of a viral vector to cells by transduction may occur in vitro, ex vivo, or in vitro. Accordingly, in some aspects of the invention, the composition that provides for the expression of a transgene in mammalian cells is a gene therapy vector, wherein the gene therapy vector comprises a polynucleotide cassette, e.g., a gene transfer cassette, of the present disclosure.

The genetic correction of HSCs from FA patients, followed by the autologous transplantation of these cells (hematopoietic gene therapy), is a good alternative for FA patients, particularly those lacking an HLA-identical sibling. In one embodiment, hematopoietic gene therapy is the preferred treatment regimen for a patient lacking an HLA-identical sibling. In another embodiment, hematopoietic gene therapy is a treatment regimen for a patient that has an HLA-identical sibling.

In particular embodiments of any of the methods disclosed herein, the gene therapy vector comprises a polynucleotide sequence encoding a FAC protein, and cells transduced with the vector are provided to a subject to treat FA.

Since most FA patients belong to the FA-A complementation group (Casado et al., 2007, Levitus et al., 2004, Taniguchi et al., 2006), a vector harboring the functional FANCA gene has been developed. The inclusion of a mutated post-transcriptional regulatory element of the woodchuck hepatitis virus (WPRE*), which lacks any residual open reading frame (Schambach A et al. Gene Ther. 2006; 13:641-645) will be used to improve the level of expression and stability of the therapeutic gene.

In some embodiments, a polynucleotide cassette comprises:
(i) a phosphoglycerate kinase (PGK) promoter sequence or a functional variant or fragment thereof;
(ii) a sequence encoding a human FANCA protein or a functional fragment or variant thereof; and:
(iii) a post-transcriptional regulatory element of the woodchuck hepatitis virus (WPRE) sequence.

In some embodiments, a polynucleotide cassette comprises:
(i) a human phosphoglycerate kinase (PGK) promoter sequence;
(ii) a sequence encoding a human FANCA protein; and:
(iii) a mutant WPRE sequence.

In some embodiments, a polynucleotide cassette comprises:
a) a 5′ LTR, optionally a modified 5′ LTR;
b) a cPPT sequence;
c) PGK promoter sequence, optionally a human PGK promoter sequence;
d) a sequence encoding a human FANCA protein, optionally a cDNA sequence or a codon optimized sequence;
e) a mutant WPRE sequence; and
f) a 3′ LTR, optionally a modified 3′ LTR.

In one embodiment, the modified WPRE is referred to as WPRE*. WPRE* is a modified WPRE that lacks an open reading frame (see, e.g., Schambach et al, 2006 Gene Ther. 13:641-645).

Since most FA patients belong to the FA-A complementation group (Casado et al., 2007, Levitus et al., 2004, Taniguchi et al., 2006), in particular embodiments, the encoded therapeutic gene product is FANCA, although the disclosure contemplates that FA proteins of other complementation groups may also be delivered, and thus encoded in the expression cassettes disclosed herein, e.g., instead of FANCA.

In particular embodiments of any of the expression cassettes and gene therapy vectors described herein, the polynucleotide sequence encoding a codon optimized FANCA is a human FANCA cDNA sequence that comprises or consists of the following sequence, or a functional fragment thereof, or a sequence having at least 80%, at least 85%, at least 90%, at least 95%, at least 98%, or at least 99% identity to the following sequence:

(SEQ ID NO: 8)
ATGTCCGACTCGTGGGTCCCGAACTCCGCCTCGGGCCAGGACCCAGGGGGCCGCCGGAGGGCCTGGGCCGAGCTG
CTGGCGGGAAGGGTCAAGAGGGAAAAATATAATCCTGAAAGGGCACAGAAATTAAAGGAATCAGCTGTGCGCCTC
CTGCGAAGCCATCAGGACCTGAATGCCCTTTTGCTTGAGGTAGAAGGTCCACTGTGTAAAAAATTGTCTCTCAGC
AAAGTGATTGACTGTGACAGTTCTGAGGCCTATGCTAATCATTCTAGTTCATTTATAGGCTCTGCTTTGCAGGAT
CAAGCCTCAAGGCTGGGGGTTCCCGTGGGTATTCTCTCAGCCGGGATGGTTGCCTCTAGCGTGGGACAGATCTGC
ACGGCTCCAGCGGAGACCAGTCACCCTGTGCTGCTGACTGTGGAGCAGAGAAAGAAGCTGTCTTCCCTGTTAGAG
TTTGCTCAGTATTTATTGGCACACAGTATGTTCTCCCGTCTTTCCTTCTGTCAAGAATTATGGAAAATACAGAGT
TCTTTGTTGCTTGAAGCGGTGTGGCATCTTCACGTACAAGGCATTGTGAGCCTGCAAGAGCTGCTGGAAAGCCAT
CCCGACATGCATGCTGTGGGATCGTGGCTCTTCAGGAATCTGTGCTGCCTTTGTGAACAGATGGAAGCATCCTGC
CAGCATGCTGACGTCGCCAGGGCCATGCTTTCTGATTTTGTTCAAATGTTTGTTTTGAGGGGATTTCAGAAAAAC
TCAGATCTGAGAAGAACTGTGGAGCCTGAAAAAATGCCGCAGGTCACGGTTGATGTACTGCAGAGAATGCTGATT
TTTGCACTTGACGCTTTGGCTGCTGGAGTACAGGAGGAGTCCTCCACTCACAAGATCGTGAGGTGCTGGTTCGGA
GTGTTCAGTGGACACACGCTTGGCAGTGTAATTTCCACAGATCCTCTGAAGAGGTTCTTCAGTCATACCCTGACT
CAGATACTCACTCACAGCCCTGTGCTGAAAGCATCTGATGCTGTTCAGATGCAGAGAGAGTGGAGCTTTGCGCGG
ACACACCCTCTGCTCACCTCACTGTACCGCAGGCTCTTTGTGATGCTGAGTGCAGAGGAGTTGGTTGGCCATTTG
CAAGAAGTTCTGGAAACGCAGGAGGTTCACTGGCAGAGAGTGCTCTCCTTTGTGTCTGCCCTGGTTGTCTGCTTT
CCAGAAGCGCAGCAGCTGCTTGAAGACTGGGTGGCGCGTTTGATGGCCCAGGCATTCGAGAGCTGCCAGCTGGAC
AGCATGGTCACTGCGTTCCTGGTTGTGCGCCAGGCAGCACTGGAGGGCCCCTCTGCGTTCCTGTCATATGCAGAC
TGGTTCAAGGCCTCCTTTGGGAGCACACGAGGCTACCATGGCTGCAGCAAGAAGGCCCTGGTCTTCCTGTTTACG
TTCTTGTCAGAACTCGTGCCTTTTGAGTCTCCCCGGTACCTGCAGGTGCACATTCTCCACCCACCCCTGGTTCCC
AGCAAGTACCGCTCCCTCCTCACAGACTACATCTCATTGGCCAAGACACGGCTGGCCGACCTCAAGGTTTCTATA
GAAAACATGGGACTCTACGAGGATTTGTCATCAGCTGGGGACATTACTGAGCCCCACAGCCAAGCTCTTCAGGAT
GTTGAAAAGGCCATCATGGTGTTTGAGCATACGGGGAACATCCCAGTCACCGTCATGGAGGCCAGCATATTCAGG
AGGCCTTACTACGTGTCCCACTTCCTCCCCGCCCTGCTCACACCTCGAGTGCTCCCCAAAGTCCCTGACTCCCGT
GTGGCGTTTATAGAGTCTCTGAAGAGAGCAGATAAAATCCCCCCATCTCTGTACTCCACCTACTGCCAGGCCTGC
TCTGCTGCTGAAGAGAAGCCAGAAGATGCAGCCCTGGGAGTGAGGGCAGAACCCAACTCTGCTGAGGAGCCCCTG
GGACAGCTCACAGCTGCACTGGGAGAGCTGAGAGCCTCCATGACAGACCCCAGCCAGCGTGATGTTATATCGGCA
CAGGTGGCAGTGATTTCTGAAAGACTGAGGGCTGTCCTGGGCCACAATGAGGATGACAGCAGCGTTGAGATATCA
AAGATTCAGCTCAGCATCAACACGCCGAGACTGGAGCCACGGGAACACATTGCTGTGGACCTCCTGCTGACGTCT
TTCTGTCAGAACCTGATGGCTGCCTCCAGTGTCGCTCCCCCGGAGAGGCAGGGTCCCTGGGCTGCCCTCTTCGTG
AGGACCATGTGTGGACGTGTGCTCCCTGCAGTGCTCACCCGGCTCTGCCAGCTGCTCCGTCACCAGGGCCCGAGC
CTGAGTGCCCCACATGTGCTGGGGTTGGCTGCCCTGGCCGTGCACCTGGGTGAGTCCAGGTCTGCGCTCCCAGAG
GTGGATGTGGGTCCTCCTGCACCTGGTGCTGGCCTTCCTGTCCCTGCGCTCTTTGACAGCCTCCTGACCTGTAGG
ACGAGGGATTCCTTGTTCTTCTGCCTGAAATTTTGTACAGCAGCAATTTCTTACTCTCTCTGCAAGTTTTCTTCC
CAGTCACGAGATACTTTGTGCAGCTGCTTATCTCCAGGCCTTATTAAAAAGTTTCAGTTCCTCATGTTCAGATTG
TTCTCAGAGGCCCGACAGCCTCTTTCTGAGGAGGACGTAGCCAGCCTTTCCTGGAGACCCTTGCACCTTCCTTCT
GCAGACTGGCAGAGAGCTGCCCTCTCTCTCTGGACACACAGAACCTTCCGAGAGGTGTTGAAAGAGGAAGATGTT
CACTTAACTTACCAAGACTGGTTACACCTGGAGCTGGAAATTCAACCTGAAGCTGATGCTCTTTCAGATACTGAA
CGGCAGGACTTCCACCAGTGGGCGATCCATGAGCACTTTCTCCCTGAGTCCTCGGCTTCAGGGGGCTGTGACGGA
GACCTGCAGGCTGCGTGTACCATTCTTGTCAACGCACTGATGGATTTCCACCAAAGCTCAAGGAGTTATGACCAC
TCAGAAAATTCTGATTTGGTCTTTGGTGGCCGCACAGGAAATGAGGATATTATTTCCAGATTGCAGGAGATGGTA
GCTGACCTGGAGCTGCAGCAAGACCTCATAGTGCCTCTCGGCCACACCCCTTCCCAGGAGCACTTCCTCTTTGAG
ATTTTCCGCAGACGGCTCCAGGCTCTGACAAGCGGGTGGAGCGTGGCTGCCAGCCTTCAGAGACAGAGGGAGCTG
CTAATGTACAAACGGATCCTCCTCCGCCTGCCTTCGTCTGTCCTCTGCGGCAGCAGCTTCCAGGCAGAACAGCCC
ATCACTGCCAGATGCGAGCAGTTCTTCCACTTGGTCAACTCTGAGATGAGAAACTTCTGCTCCCACGGAGGTGCC
CTGACACAGGACATCACTGCCCACTTCTTCAGGGGCCTCCTGAACGCCTGTCTGCGGAGCAGAGACCCCTCCCTG
ATGGTCGACTTCATACTGGCCAAGTGCCAGACGAAATGCCCCTTAATTTTGACCTCTGCTCTGGTGTGGTGGCCG
AGCCTGGAGCCTGTGCTGCTCTGCCGGTGGAGGAGACACTGCCAGAGCCCGCTGCCCCGGGAACTGCAGAAGCTA
CAAGAAGGCCGGCAGTTTGCCAGCGATTTCCTCTCCCCTGAGGCTGCCTCCCCAGCACCCAACCCGGACTGGCTC
TCAGCTGCTGCACTGCACTTTGCGATTCAACAAGTCAGGGAAGAAAACATCAGGAAGCAGCTAAAGAAGCTGGAC
TGCGAGAGAGAGGAGCTATTGGTTTTCCTTTTCTTCTTCTCCTTGATGGGCCTGCTGTCGTCACATCTGACCTCA
AATAGCACCACAGACCTGCCAAAGGCTTTCCACGTTTGTGCAGCAATCCTCGAGTGTTTAGAGAAGAGGAAGATA
TCCTGGCTGGCACTCTTTCAGTTGACAGAGAGTGACCTCAGGCTGGGGCGGCTCCTCCTCCGTGTGGCCCCGGAT
CAGCACACCAGGCTGCTGCCTTTCGCTTTTTACAGTCTTCTCTCCTACTTCCATGAAGACGCGGCCATCAGGGAA
GAGGCCTTCCTGCATGTTGCTGTGGACATGTACTTGAAGCTGGTCCAGCTCTTCGTGGCTGGGGATACAAGCACA
GTTTCACCTCCAGCTGGCAGGAGCCTGGAGCTCAAGGGTCAGGGCAACCCCGTGGAACTGATAACAAAAGCTCGT
CTTTTTCTGCTGCAGTTAATACCTCGGTGCCCGAAAAAGAGCTTCTCACACGTGGCAGAGCTGCTGGCTGATCGT
GGGGACTGCGACCCAGAGGTGAGCGCCGCCCTCCAGAGCAGACAGCAGGCTGCCCCTGACGCTGACCTGTCCCAG
GAGCCTCATCTCTTCTGA;
or
(SEQ ID NO: 36)
ATGTCCGACTCGTGGGTCCCGAACTCCGCCTCGGGCCAGGACCCAGGGGGCCGCCGGAGGGCCTGGGCCGAGCTG
CTGGCGGGAAGGGTCAAGAGGGAAAAATATAATCCTGAAAGGGCACAGAAATTAAAGGAATCAGCTGTGCGCCTC
CTGCGAAGCCATCAGGACCTGAATGCCCTTTTGCTTGAGGTAGAAGGTCCACTGTGTAAAAAATTGTCTCTCAGC
AAAGTGATTGACTGTGACAGTTCTGAGGCCTATGCTAATCATTCTAGTTCATTTATAGGCTCTGCTTTGCAGGAT
CAAGCCTCAAGGCTGGGGGTTCCCGTGGGTATTCTCTCAGCCGGGATGGTTGCCTCTAGCGTGGGACAGATCTGC
ACGGCTCCAGCGGAGACCAGTCACCCTGTGCTGCTGACTGTGGAGCAGAGAAAGAAGCTGTCTTCCCTGTTAGAG
TTTGCTCAGTATTTATTGGCACACAGTATGTTCTCCCGTCTTTCCTTCTGTCAAGAATTATGGAAAATACAGAGT
TCTTTGTTGCTTGAAGCGGTGTGGCATCTTCACGTACAAGGCATTGTGAGCCTGCAAGAGCTGCTGGAAAGCCAT
CCCGACATGCATGCTGTGGGATCGTGGCTCTTCAGGAATCTGTGCTGCCTTTGTGAACAGATGGAAGCATCCTGC
CAGCATGCTGACGTCGCCAGGGCCATGCTTTCTGATTTTGTTCAAATGTTTGTTTTGAGGGGATTTCAGAAAAAC
TCAGATCTGAGAAGAACTGTGGAGCCTGAAAAAATGCCGCAGGTCACGGTTGATGTACTGCAGAGAATGCTGATT
TTTGCACTTGACGCTTTGGCTGCTGGAGTACAGGAGGAGTCCTCCACTCACAAGATCGTGAGGTGCTGGTTCGGA
GTGTTCAGTGGACACACGCTTGGCAGTGTAATTTCCACAGATCCTCTGAAGAGGTTCTTCAGTCATACCCTGACT
CAGATACTCACTCACAGCCCTGTGCTGAAAGCATCTGATGCTGTTCAGATGCAGAGAGAGTGGAGCTTTGCGCGG
ACACACCCTCTGCTCACCTCACTGTACCGCAGGCTCTTTGTGATGCTGAGTGCAGAGGAGTTGGTTGGCCATTTG
CAAGAAGTTCTGGAAACGCAGGAGGTTCACTGGCAGAGAGTGCTCTCCTTTGTGTCTGCCCTGGTTGTCTGCTTT
CCAGAAGCGCAGCAGCTGCTTGAAGACTGGGTGGCGCGTTTGATGGCCCAGGCATTCGAGAGCTGCCAGCTGGAC
AGCATGGTCACTGCGTTCCTGGTTGTGCGCCAGGCAGCACTGGAGGGCCCCTCTGCGTTCCTGTCATATGCAGAC
TGGTTCAAGGCCTCCTTTGGGAGCACACGAGGCTACCATGGCTGCAGCAAGAAGGCCCTGGTCTTCCTGTTTACG
TTCTTGTCAGAACTCGTGCCTTTTGAGTCTCCCCGGTACCTGCAGGTGCACATTCTCCACCCACCCCTGGTTCCC
AGCAAGTACCGCTCCCTCCTCACAGACTACATCTCATTGGCCAAGACACGGCTGGCCGACCTCAAGGTTTCTATA
GAAAACATGGGACTCTACGAGGATTTGTCATCAGCTGGGGACATTACTGAGCCCCACAGCCAAGCTCTTCAGGAT
GTTGAAAAGGCCATCATGGTGTTTGAGCATACGGGGAACATCCCAGTCACCGTCATGGAGGCCAGCATATTCAGG
AGGCCTTACTACGTGTCCCACTTCCTCCCCGCCCTGCTCACACCTCGAGTGCTCCCCAAAGTCCCTGACTCCCGT
GTGGCGTTTATAGAGTCTCTGAAGAGAGCAGATAAAATCCCCCCATCTCTGTACTCCACCTACTGCCAGGCCTGC
TCTGCTGCTGAAGAGAAGCCAGAAGATGCAGCCCTGGGAGTGAGGGCAGAACCCAACTCTGCTGAGGAGCCCCTG
GGACAGCTCACAGCTGCACTGGGAGAGCTGAGAGCCTCCATGACAGACCCCAGCCAGCGTGATGTTATATCGGCA
CAGGTGGCAGTGATTTCTGAAAGACTGAGGGCTGTCCTGGGCCACAATGAGGATGACAGCAGCGTTGAGATATCA
AAGATTCAGCTCAGCATCAACACGCCGAGACTGGAGCCACGGGAACACATTGCTGTGGACCTCCTGCTGACGTCT
TTCTGTCAGAACCTGATGGCTGCCTCCAGTGTCGCTCCCCCGGAGAGGCAGGGTCCCTGGGCTGCCCTCTTCGTG
AGGACCATGTGTGGACGTGTGCTCCCTGCAGTGCTCACCCGGCTCTGCCAGCTGCTCCGTCACCAGGGCCCGAGC
CTGAGTGCCCCACATGTGCTGGGGTTGGCTGCCCTGGCCGTGCACCTGGGTGAGTCCAGGTCTGCGCTCCCAGAG
GTGGATGTGGGTCCTCCTGCACCTGGTGCTGGCCTTCCTGTCCCTGCGCTCTTTGACAGCCTCCTGACCTGTAGG
ACGAGGGATTCCTTGTTCTTCTGCCTGAAATTTTGTACAGCAGCAATTTCTTACTCTCTCTGCAAGTTTTCTTCC
CAGTCACGAGATACTTTGTGCAGCTGCTTATCTCCAGGCCTTATTAAAAAGTTTCAGTTCCTCATGTTCAGATTG
TTCTCAGAGGCCCGACAGCCTCTTTCTGAGGAGGACGTAGCCAGCCTTTCCTGGAGACCCTTGCACCTTCCTTCT
GCAGACTGGCAGAGAGCTGCCCTCTCTCTCTGGACACACAGAACCTTCCGAGAGGTGTTGAAAGAGGAAGATGTT
CACTTAACTTACCAAGACTGGTTACACCTGGAGCTGGAAATTCAACCTGAAGCTGATGCTCTTTCAGATACTGAA
CGGCAGGACTTCCACCAGTGGGCGATCCATGAGCACTTTCTCCCTGAGTCCTCGGCTTCAGGGGGCTGTGACGGA
GACCTGCAGGCTGCGTGTACCATTCTTGTCAACGCACTGATGGATTTCCACCAAAGCTCAAGGAGTTATGACCAC
TCAGAAAATTCTGATTTGGTCTTTGGTGGCCGCACAGGAAATGAGGATATTATTTCCAGATTGCAGGAGATGGTA
GCTGACCTGGAGCTGCAGCAAGACCTCATAGTGCCTCTCGGCCACACCCCTTCCCAGGAGCACTTCCTCTTTGAG
ATTTTCCGCAGACGGCTCCAGGCTCTGACAAGCGGGTGGAGCGTGGCTGCCAGCCTTCAGAGACAGAGGGAGCTG
CTAATGTACAAACGGATCCTCCTCCGCCTGCCTTCGTCTGTCCTCTGCGGCAGCAGCTTCCAGGCAGAACAGCCC
ATCACTGCCAGATGCGAGCAGTTCTTCCACTTGGTCAACTCTGAGATGAGAAACTTCTGCTCCCACGGAGGTGCC
CTGACACAGGACATCACTGCCCACTTCTTCAGGGGCCTCCTGAACGCCTGTCTGCGGAGCAGAGACCCCTCCCTG
ATGGTCGACTTCATACTGGCCAAGTGCCAGACGAAATGCCCCTTAATTTTGACCTCTGCTCTGGTGTGGTGGCCG
AGCCTGGAGCCTGTGCTGCTCTGCCGGTGGAGGAGACACTGCCAGAGCCCGCTGCCCCGGGAACTGCAGAAGCTA
CAAGAAGGCCGGCAGTTTGCCAGCGATTTCCTCTCCCCTGAGGCTGCCTCCCCAGCACCCAACCCGGACTGGCTC
TCAGCTGCTGCACTGCACTTTGCGATTCAACAAGTCAGGGAAGAAAACATCAGGAAGCAGCTAAAGAAGCTGGAC
TGCGAGAGAGAGGAGCTATTGGTTTTCCTTTTCTTCTTCTCCTTGATGGGCCTGCTGTCGTCACATCTGACCTCA
AATAGCACCACAGACCTGCCAAAGGCTTTCCACGTTTGTGCAGCAATCCTCGAGTGTTTAGAGAAGAGGAAGATA
TCCTGGCTGGCACTCTTTCAGTTGACAGAGAGTGACCTCAGGCTGGGGCGGCTCCTCCTCCGTGTGGCCCCGGAT
CAGCACACCAGGCTGCTGCCTTTCGCTTTTTACAGTCTTCTCTCCTACTTCCATGAAGACGCGGCCATCAGGGAA
GAGGCCTTCCTGCATGTTGCTGTGGACATGTACTTGAAGCTGGTCCAGCTCTTCGTGGCTGGGGATACAAGCACA
GTTTCACCTCCAGCTGGCAGGAGCCTGGAGCTCAAGGGTCAGGGCAACCCCGTGGAACTGATAACAAAAGCTCGT
CTTTTTCTGCTGCAGTTAATACCTCGGTGCCCGAAAAAGAGCTTCTCACACGTGGCAGAGCTGCTGGCTGATCGT
GGGGACTGCGACCCAGAGGTGAGCGCCGCCCTCCAGAGCAGACAGCAGGCTGCCCCTGACGCTGACCTGTCCCAG
GAGCCTCATCTCTTCTGATGA.

The present disclosure includes plasmids comprising an expression cassette or transfer cassette described herein. In particular embodiments, the plasmid is pCCL-PGK-FANCA-WPRE* or pCCL-PGK-FANCAW-82-RO (SEQ ID NO: 25).

In some embodiments, the gene therapy vector is a self-limiting LV. In a specific embodiment of any of the expression cassettes and gene therapy vectors described herein, the transfer cassette is a pCCL-SIN-cPPT/CTS-hPGK-hFANCA-WPRE of the disclosure comprises or consists of the following sequence, or a sequence having at least 80%, at least 85%, at least 90%, at least 95%, at least 98%, or at least 99% identity to SEQ ID NO: 24. SEQ ID NO: 24 corresponds to the pCCL-PGK-FANCA-WPRE* plasmid. SEQ ID NO: 25 corresponds to the pCCL-PGK-FANCAW-82-RO plasmid.

In one embodiment, a FANCA gene is delivered via a lentiviral vector (LV). The FANCA LVs described herein utilize a self-inactivating lentiviral vector (LV). In one embodiment, the FANCA LV comprises a promoter of the human phosphoglycerate (PGK) gene. The safety properties of this vector have been markedly improved, compared to the gamma-retroviral vectors already used in the clinics, which harbored strong viral promoters.

In certain embodiments, the lentiviral vector is PGK-FANCA.WPRE*LV, which comprises the gene transfer cassette, comprising sequences disclosed in SEQ ID NO: 24. The PGK-FANCA-WPRE*LV gene expression cassette portion comprises the human PGK promoter, the coding sequence for FANCA cDNA, and the WPRE*; and corresponds to nucleotides 3541 to 9178 of SEQ ID NO: 24. The PGK-FANCA-WPRE*LV transfer cassette portion comprises from about the 5′ LTR (U5) to about the 3′ LTR (U5) of the sequence. With respect to SEQ ID NO: 24, nucleotides 1586-1789 of SEQ ID NO: 24 comprise human CMV immediate early promoter. Nucleotides 2031-2156 of SEQ ID NO: 24 comprise HIV1 psi packaging signal. Nucleotides 2649-2882 of SEQ ID NO: 24 comprise HIV1 RRE element. Nucleotides 3378-3495 of SEQ ID NO: 24 comprise HIV cPPT/CTS element. Nucleotides 3541-4051 of SEQ ID NO: 24 comprise the hPGK promoter. Nucleotides 4078-8445 of SEQ ID NO: 24 comprise human FANCA-A cDNA. Nucleotides 8502-9178 of SEQ ID NO: 24 comprise mutated WPRE element. Nucleotides 9262-9495 of SEQ ID NO: 24 comprise the HIV delta U 3′ LTR.

In certain embodiments, the lentiviral vector is pCCL-PGK-FANCAW-82-RO, which comprises the gene transfer cassette, comprising sequences disclosed in SEQ ID NO: 25. The pCCL-PGK-FANCAW-82-RO gene expression cassette portion comprises the human PGK promoter, the coding sequence for FANCA cDNA, and the WPRE*. The pCCL-PGK-FANCAW-82-RO transfer cassette portion comprises from about the 5′ LTR (U5) to about the 3′ LTR (U5) of the sequence. Nucleotides 1-511 of SEQ ID NO: 25 comprise the hPGK promoter. Nucleotides 538-4905 of SEQ ID NO: 25 comprise human FANCA-A cDNA.

In yet another embodiment, the lentiviral vector contains the following elements: (i) the backbone of the lentiviral vector derived from the initial pCCLsin-cppt-hPGK-eGFP-WPRE (Dull et al, 1998; J. Virol 72 (11), 9873-9880). The pCCL backbone utilizes a heterologous CMV-HIV 5′ LTR to obtain high levels of viral RNA transcription in the producer cells. Such heterologous LTR renders the construct independent from the need to use the HIV Tat protein for the production of the rHIV particles and it is therefore a safety feature. The U3 region of the 3′ LTR contains a 400 bp deletion as described in (Zufferey et al J Virol, 1998) which confers self-inactivating properties to the vector; (ii) the cDNA of the human codon optimized FANCA gene (4368 bp GenBank accession number: X_99226 or as disclosed herein) encoding the FANCA protein (1455 AA) under control of the human PGK promoter. The promoter has already been characterized by its stable activity in vivo and by improved safety properties, compared to other promoters already used in gene therapy; and (iii) a mutated version of the woodchuck hepatitis virus post-transcriptional regulatory element (WPRE) that is deleted in the 3′ region of a sequence coding for the X protein and any residual ORF of the described by Schambach et al (Gene therapy, 2006; 13, 641-645) or WPRE*.

In certain embodiments, the FANCA LVs described herein utilize a self-inactivating lentiviral vector (LV). In one embodiment, the FANCA LV comprises a promoter of the human phosphoglycerate (PGK) gene. The safety properties of this vector have been markedly improved, compared to the gamma-retroviral vectors already used in the clinics, which harbored strong viral promoters. In one embodiment, a FANCA gene is delivered via a lentiviral vector. In certain embodiments, the lentiviral vector is PGK-FANCA.WPRE*LV.

Gene therapy vectors encapsulating the polynucleotide cassettes of the present disclosure may be produced using standard methodology. For example, in the case of LV virions, an LV expression vector according to the invention may be introduced into a producer cell, followed by introduction of an LV helper construct, where the helper construct includes LV coding regions capable of being expressed in the producer cell and which complement LV helper functions absent in the LV vector. This is followed by introduction of helper virus and/or additional vectors into the producer cell, wherein the helper virus and/or additional vectors provide accessory functions capable of supporting efficient LV virus production. The producer cells are then cultured to produce LV. These steps are carried out using standard methodology. In particular embodiments, the plasmids depicted in FIG. 1 is used to produce the gene therapy vectors.

Any suitable method for producing viral vector particles for delivery of the subject polynucleotide cassettes can be used, including but not limited to those described in the examples that follow. Any concentration of infective viral vector particles suitable to effectively transduce mammalian cells can be prepared for contacting mammalian cells in vitro or in vivo. For example, the viral particles may be formulated at a concentration of 10⁸ infectious units per ml or more, for example, 5×10⁸ infectious units per mL; 10⁹ infectious units per mL; 5×10⁹ infectious units per mL, 10¹⁰ infectious units per mL, 5×10¹⁰ infectious units per mL; 10¹¹ infectious units per mL; 5×10¹¹ infectious units per mL; 10¹² infectious units per mL; 5×10¹² infectious units per mL; 10¹³ infectious units per mL; 1.5×10¹³ infectious units per mL; 3×10¹³ infectious units per mL; 5×10¹³ infectious units per mL; 7.5×10¹³ infectious units per mL; 9×10¹³ infectious units per mL; 1×10¹⁴ infectious units per mL, 5×10¹⁴ infectious units per mL or more, but typically not more than 1×10¹⁵ infectious units per mL.

In preparing the subject LV gene therapy vectors, any host cells for producing LV virions may be employed, including, for example, mammalian cells (e.g. 293 cells), insect cells (e.g. SF9 cells), microorganisms and yeast. Host cells can also be packaging cells in which the LV rep and cap genes are stably maintained in the host cell or producer cells in which the LV vector genome is stably maintained and packaged. Exemplary packaging and producer cells are derived from SF-9, 293, A549 or HeLa cells. LV vectors are purified and formulated using standard techniques known in the art.

In certain embodiments, the present invention includes a cell comprising a gene expression cassette, gene transfer cassette, or gene therapy vector disclosed herein. In related embodiments, the cell is transduced with a gene therapy vector comprising an expression cassette disclosed herein or has an expression cassette disclosed herein integrated into the cell's genome. In certain embodiments, the cell is a cell used to produce a viral gene therapy vector, e.g., a packaging cell.

In other embodiments, the cell is a cell to be delivered to a subject in order to provide to the subject the gene product encoded by the expression cassette. Thus, in certain embodiments, the cell is autologous to the subject to be treated or was obtained from the subject to be treated. In other embodiments, the cell is allogeneic to the subject to be treated or was obtained from a donor other than the subject to be treated. In particular embodiments, the cell is a mammalian cell, e.g., a human cell. In certain embodiments, the cell is a blood cell, an erythrocyte, a hematopoietic progenitor cell, a bone marrow cell, e.g., a lineage depleted bone marrow cell, a hematopoietic stem cell (e.g., CD34+) or a committed hematopoietic erythroid progenitor cell. In particular embodiments, the cell is a CD34+ cell obtained from a subject to be treated with the cell after it is transduced by a gene therapy vector disclosed herein. In particular embodiment, the cell is a CD34+FA cell obtained from a subject diagnosed with FA.

In some embodiments, the methods disclosed herein result in a therapeutic benefit, e.g., preventing the development of a disorder, halting the progression of a disorder, reversing the progression of a disorder, etc. For example, in one embodiment, the disorder is BMF. In one embodiment, the disorder is thrombocytopenia. In another embodiment, the disorder is leukopenia. In one embodiment, the disorder is pancytopenia. In one embodiment, the disorder is neutropenia. In another embodiment, the disorder is anemia. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

In another embodiment, the present invention includes a method of treating a disease in a subject in need thereof comprising providing to the subject an effective amount of either or both of a high-stringency CD34-enriched cell population and a low-stringency CD34-enriched cell population, either or both of which have been contacted with a gene therapy vector, e.g., a viral vector, that expresses a therapeutic gene product in the cells. In particular embodiments, the either or both of a high-stringency CD34-enriched cell population and a low-stringency CD34-enriched cell population are autologous to the subject. In certain embodiments, the cells are erythroid cells, e.g., hematopoietic stem cells or committed hematopoietic erythroid progenitor cells. In some embodiments, the cell is a bone marrow cell, e.g., a lineage depleted bone marrow cell. In particular embodiments, the method is used to treat FA, and the viral vector is a LV comprising an expression construct disclosed herein comprising a human PGK promoter operably linked to a FANCA gene cDNA or coding sequence, and a mutated wPRE disclosed herein. In particular embodiments, the cells are provided to the subject parenterally, e.g., via intravenous injection.

In another embodiment, the present invention includes a method of treating FA in a subject in need thereof, comprising providing to the subject an effective amount of autologous high-stringency CD34-enriched cell population and/or a low-stringency CD34-enriched cell populations of stem cells transduced with a LV vector that expresses a FANCA cDNA in the cells, wherein the LV vector comprises a human PGK promoter operably linked to the FANCA cDNA or coding sequence, and a mutated wPRE sequence disclosed herein. In particular embodiments, the cells are hematopoietic stem cells or committed hematopoietic erythroid progenitor cells, e.g., bone marrow cells. In particular embodiments, the cells are provided to the subject parenterally, e.g., via intravenous injection.

Expression of the transgene using the subject transgene is expected to be robust. Accordingly, in some instances, the expression of the transgene, e.g. as detected by measuring levels of gene product, by measuring therapeutic efficacy, etc. may be observed two months or less after administration, e.g. 4, 3 or 2 weeks or less after administration, for example, 1 week after administration of the subject composition. Expression of the transgene is also expected to persist over time. Accordingly, in some instances, the expression of the transgene, e.g. as detected by measuring levels of gene product, by measuring therapeutic efficacy, etc., may be observed 2 months or more after administration of the subject composition, e.g., 4, 6, 8, or 10 months or more, in some instances 1 year or more, for example 2, 3, 4, or 5 years, in certain instances, more than 5 years.

In certain embodiments, the method comprises the step of detecting expression of the transgene in the cells or in the subject, wherein expression is enhanced relative to expression from a polynucleotide cassette not comprising the one or more improved elements of the present disclosure. Typically, expression will be enhanced 2-fold or more relative to the expression from a reference, i.e. a control polynucleotide cassette, e.g. as known in the art, for example 3-fold, 4-fold, or 5-fold or more, in some instances 10-fold, 20-fold or 50-fold or more, e.g. 100-fold, as evidenced by, e.g. earlier detection, higher levels of gene product, a stronger functional impact on the cells, etc.

In some embodiments, the dose of cells patients receive by infusion will be that which is obtained from the transduction process. In various preferred embodiments, at least about 1×10¹, 1×10², 1×10³, 1×10⁴, 1×10⁵, 1×10⁶, 1×10⁷, 1×10⁸, or more high-stringency CD34-enriched cells/KG of patient weight are infused into the patient. In various preferred embodiments, at least at least about 1×10¹, 1×10², 1×10³, 1×10⁴, 1×10⁵, 1×10⁶, 1×10⁷, 1×10⁸′ or more low-stringency CD34-enriched cells/KG of patient weight are infused into the patient. In some embodiments, between 1×10⁶ and 4×10⁶ high-stringency CD34-enriched cells/Kg of patient weight are infused into the patient. In other embodiments, 3×10⁵ and 4×10⁶ high-stringency CD34-enriched cells/Kg of patient weight are infused into the patient. In some embodiments, between 1×10⁶ and 4×10⁶ high-stringency CD34-enriched cells/Kg of patient weight are infused into the patient. In other embodiments, 3×10⁵ and 4×10⁶ high-stringency CD34-enriched cells/Kg of patient weight are infused into the patient. In some embodiments, cells will be infused into the patient a single dose. In other embodiments, cells will be infused into the patient in multiple doses (e.g, the high-stringency and low-stringency CD34-enriched cell populations are administered sequentially once or multiple times). Transduced cells may be infused immediately after the transduction process is completed. In particular embodiments, the transduced cells are stored or frozen before use, whereas in certain embodiments, they are provided to the subject immediately or shortly after they are transduced, e.g., within one hour, two hours, four hours, or eight hours.

Once integrated, the therapeutic protein (e.g., human FANCA protein) is expressed by the cells. Transduced FA cells are genetically corrected, and thus able to activate the FA pathway by the mono-ubiquitination of FANCD2 and FANCI. These proteins migrate to areas of DNA damage, and in cooperation with other DNA repair proteins, promote the repair of the DNA in these cells, as occurs in healthy cells

As described in further detail in the Examples, preclinical in vitro data with BM samples from human FA patients has already shown the efficacy of an FANCA LV to correct the phenotype of these cells.

In one embodiment, at least 1 to 4×10⁶ CD34⁺ corrected cells (e.g., FANCA transduced HSCs) per kilogram of patient weight are administered, e.g., to restore hematopoiesis in a non-conditioned FA patient. In some embodiments, the transduced cells are infused or administered into the patient immediately after transduction. In other embodiments, the transduced cells are frozen prior to infusing or administering into the patient

The genetic correction of HSCs from FA patients, followed by the autologous transplantation of these cells (hematopoietic gene therapy), is a good alternative for FA patients, particularly those lacking an HLA-identical sibling. In one embodiment, hematopoietic gene therapy is the preferred treatment regimen for a patient lacking an HLA-identical sibling. In another embodiment, hematopoietic gene therapy is the preferred treatment regimen for a patient that has an HLA-identical sibling.

### Compositions and Formulations

Certain aspects of the disclosure relate to a system or combination of a high-stringency CD34-enriched cell population and a low-stringency CD34-enriched cell population, either or both of which have been transduced with a gene therapy vector. Some embodiments comprise a combination of high-stringency CD34-enriched and low-stringency CD34-enriched cell populations, either or both of which have been transduced with a lentiviral vector containing a human FANC gene, e.g. FANCA, FANCC, or FANCG, or a nucleic acid sequence encoding a FANC protein, including functional variants and fragments thereof. Some embodiments comprise a combination of high-stringency CD34-enriched and low-stringency CD34-enriched cell populations either or both of which have been subjected to gene editing or gene repair, such as with a CRISPR, TALEN, zinc-finger, or meganuclease gene editing system. Some embodiments comprises a combination of high-stringency CD34-enriched and low-stringency CD34-enriched cell populations that has been transduced with a lentiviral (or other viral) vector containing a transgene associated with a disease or condition, such as an immunodeficiency disorder.

In some aspects of the disclosure, formulations are provided for the treatment of a disease or condition. The formulations may comprise high-stringency CD34-enriched cell population(s) or low-stringency CD34-enriched cell population(s) or both, along with a physiologically acceptable carrier or pharmaceutically acceptable carrier as described herein. The formulations may comprise high-stringency CD34-enriched cell population(s) or low-stringency CD34-enriched cell population(s) or both, wherein one or both cell populations were transduced with a gene therapy vector and express or are capable of expressing a therapeutic agent, along with a physiologically acceptable carrier or pharmaceutically acceptable carrier as described herein.

The present invention includes pharmaceutical compositions and formulations comprising either or both of a high-stringency CD34-enriched cell population and a low-stringency CD34-enriched cell population gene therapy vector as described herein and a pharmaceutically-acceptable carrier, diluent or excipient. The subject high-stringency CD34-enriched cell population and/or a low-stringency CD34-enriched cell population can be combined with pharmaceutically-acceptable carriers, diluents and reagents useful in preparing a formulation that is generally safe, non-toxic, and desirable, and includes excipients that are acceptable for primate use. Examples of such excipients, carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, dimethyl sulfoxide (DMSO), fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the formulations are sterile.

In some embodiments, the CD34-enriched cell populations are manufactured in accordance with Current Good Manufacturing Practices. Manufactured in accordance with Current Good Manufacturing Practices means that the formulation prepared for administration is sufficiently safe to permit administration to a human subject under controlling regulations and government authorizations. Generally, the controlling regulations and authorizations will dictate that the formulation meet pre-approved acceptance criteria regarding identity, strength, quality and purity. Acceptance criteria include numerical limits, ranges, or other suitable measures of test results used to determine whether a formulation meets the Current Good Manufacturing Practices. A specification sets forth the analytical procedures that are used to test conformance with the acceptance criteria. Formulations can be assessed in batches. A batch is a specific quantity of a formulation tested to ensure compliance with acceptance criteria.

The formulations can be included in a container, pack, or dispenser, e.g. syringe, e.g. a prefilled syringe, together with instructions for administration.

Where necessary or beneficial, formulations can include a local anesthetic such as lidocaine to ease pain at a site of injection.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

The formulations disclosed herein can be prepared for administration by, for example, injection, infusion, perfusion, or lavage. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹. In particular embodiments, a minimum dose is 2×10⁶ cells/kg subject body weight.

In some embodiments, the pharmaceutical composition provided herein comprise a therapeutically effective amount of either or both of a high-stringency CD34-enriched cell population and a low-stringency CD34-enriched cell populations disclosed herein in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed.

Because FA-A is the most frequent complementation group in FA patients (Casado et al., 2007, Taniguchi et al., 2006), vectors expressing the FANCA gene and/or the EGFP marker gene are the focus of the Examples; however, other FANCA genes may be utilized to similarly treat other complementation groups

The disclosure is further described in the following Examples, which do not limit the scope of the disclosure described in the claims EXAMPLES

### Example 1

### Treatment of Patient with High- and Low-Stringency CD34-Enriched Cell Populations

Patient 1 presented with Fanconi anemia. Patient 1 thereafter underwent mobilization using G-CSF and plerixafor followed by two apheresis collections on successive days. Relative to other FA patients on the same clinical trial, Patient 1 had moderate mobilization of CD34 cells, indicated by peripheral blood analysis of circulating CD34 cells, with similar mobilization kinetics to other patients. Following two apheresis collections, collected HSPCs were divided into two peripheral-blood biological samples.

High-stringency CD34 selection: The first biological sample was enriched by selection for CD34+ cells under high-stringency conditions using Miltenyi Biotec CliniMACS® system in enrichment mode with Miltenyi Biotec CD34 Reagent for enrichment, as described in U.S. Pat. No. 8,727,132. High-stringency CD34 selection resulted in 29.0% yield of CD34⁺ cells and 36% relative purity.

Low-stringency CD34 selection: The second biological sample was enriched by selection for CD34+ cells under low-stringency conditions using a modification of depletion mode use of the Miltenyi Biotec CliniMACS® system. Briefly, the second biological sample was labelled using Miltenyi Biotec CD34 Reagant. The sample was then loaded on the column using the depletion mode program for the instrument. After bulk loading of the sample with the magnet “ON,” the Cell Collection Bag (used in normal operation of the instrument to collect target cells) was removed and not used for stem-cell transplant. The “Non-Target Cell Bag” was attached, then the magnet was turned “OFF,” and then elution buffer was applied to the instrument resulting in elution of CD34⁺ cells into the Non-Target Cell Bag. The cell population collected in the Non-Target Cell Bag was kept and designated as the low-stringency CD34-enriched cell population. Low-stringency CD34 selection resulted in 54.5% yield of CD34⁺ cells at 5.8% relative purity. The low purity is due to other hematopoietic cell types that were not purified away from the CD34⁺ cells during the selection process. Results are summarized in Tables 1A and 1B.

TABLE 1A
Before CD34 Enrichment

| | | | | Total | |
|---|---|---|---|---|---|
| FA | Aph | TNC | CD34% | CD34 | CD34/kg |
| Pt #1 | 1 | 1.36E+10 | 0.15 | 2.04E+07 | 1.46E+06 |
| | 2 | 2.30E+10 | 0.1 | 2.30E+07 | 1.64E+06 |

TABLE 1B
After CD34 Enrichment

| | | | After CD34 Enrichment | | | | |
|---|---|---|---|---|---|---|---|
| | | CD34- | | | Total | | CD34 |
| FA | Aph | Enrich. | TNC | CD34% | CD34 | CD34/kg | Yield |
| Pt | 1 | Low | 1.92E+08 | 5.8 | 1.11E+07 | 7.95E+05 | 54.5 |
| #1 | 2 | High | 1.85E+07 | 36 | 6.66E+06 | 4.76E+05 | 29.0 |

Starting cell numbers and CD34+ cell purity and resulting cell populations from a Fanconi Anemia-A patient
Aph: Apheresis
TNC: Total Nucleated Cell Count
Low: Modified CliniMACS depletion program
High: Standard CliniMACS CD34 enrichment program

Subsequently, both high-stringency CD34-enriched cell population and low-stringency CD34-enriched cell population were each individually transduced with a recombinant gene therapy vector (PGK-FANCA-WPRE*) (SEQ ID NO: 25) encoding the FANCA gene product, and the resulting genetically modified cells were designated as Product 1.1 and Product 1.2, respectively.

Product 1.1 and Product 1.2 were mixed together and thereafter administered to Patient 1 by continuous intravenous infusion over 10-30 minutes while monitoring clinical indicators.

Patient 1 has shown the most rapid, or nearly the most rapid, in vivo selection engraftment kinetics of all FA patients transplanted at participating institutions. Early trends of stabilization of hematopoietic lineages that had declined in this FA patient prior to transplantation is a convincing endpoint for clinical success, which was unexpectedly achieved by this treatment method. Five other FA patients have been transplanted with standard CD34 selection approaches and none have shown comparable engraftment kinetics to Patient 1.

Without being bound by theory, it is believed that the mixture of high-stringency (Product 1.1) and low-stringency (Product 1.2) CD34-enriched cell populations confers an in vivo selective advantage to gene-modified FA hematopoietic cells and results in a progressive increase in gene-modified cells over time. Product 1.1 (high-stringency CD34-enriched cell population) is thought to be providing the majority of the gene-modified cells contributing to hematopoiesis and Product 1.2 (low-stringency CD34-enriched cell population) is thought to have facilitated robust engraftment and hematopoietic repopulation by Product 1.1.

### Example 2

### Comparative Efficacy of Treatment with Lineage-Depletion Cell Population

Patient 2 presented with Fanconi anemia. Peripheral blood biological sample were obtained from Patient 2 by mobilized apheresis using equivalent procedures to those of Example 1. A lineage-depletion cell population was prepared by labelling the biological sample with CD3/CD14/CD16/CD19 reagent and depleting the sample of labelled cells using the Miltenyi Biotec CliniMACS® system in depletion mode. Lineage-depletion CD34 selection results in 56% yield of CD34⁺ cells at 1.6% relative purity. The low purity is due to other hematopoietic cell types that were not purified away from the CD34⁺ cells during the selection process. CD34 cell yield and purity were comparable to those achieved with Patient 1 in the low-stringency CD34-enriched cell population. No high-stringency CD34-enriched cell population was prepared. The lineage-depleted cell population was transduced with a recombinant gene therapy vector encoding the FANCA gene product, and designated as Product 2.1.

Product 2.1 administered to Patient 2 by continuous intravenous infusion over 10-30 minutes while monitoring clinical indicators in cell numbers comparable to those used with Patient 1. In contrast to Patient 1, no gene-modified cells could be detected in the blood of Patient 2 six months after transplantation. Thus, Product 2.1, believed to be similar to Product 1.2 alone, results in no detectable hematopoietic recovery.

### Example 3

### Modification of Low Stringency Conditions for Higher Yield and Treatment with Resulting Cell Populations

Patient 3 presents with Fanconi anemia. Peripheral blood biological sample are obtained from Patient 3 by mobilized apheresis using equivalent procedures to those of Example 1. Following apheresis collection, collected HSPCs are divided into two peripheral-blood biological samples.

High-stringency CD34 selection: A first biological sample is enriched by selection for CD34+ cells under high-stringency conditions using Miltenyi Biotec CliniMACS® system in enrichment mode with Miltenyi Biotec CD34 Reagent for enrichment, as described in Example 1. High-stringency CD34 selection results in greater than 20% yield of CD34⁺ cells and greater than 20% relative purity. The CD34 Selection Program on a CliniMACS® system is run using the sample as described above with the parameters as indicated in Table 2. The process lasts approximately 20-30 minutes. The “target cell bag” of the CliniMACS® system contains the high-stringency CD34-enriched cell population.

TABLE 2

| Input Program and Sample Parameters | |
|---|---|
| Separation Program | ENRICHMENT 1.1 |
| Tubing Set (select from list) | CliniMACS LS Tubing |
| | Set REF 162-01 |
| Cell Concentration [*10°/mL] | 180 \| Min: 20, Max: 400 |
| Frequency of Labelled Cells [%] | 1 \| Min: 1, Max: 80 |
| Sample Loading Volume [mL] | 150 \| Min: 60, Max: 400 |
| Calculated Process Parameters | |
| Max. Processing Time [min] | 29 |
| Labelled Cells to be Processed [—] | 2.7E+08 |
| Number of Stages [—] | 1 |
| Process Specifications | Inside Specifications |
| Calculated Process Parameters- | |
| Screen Output | |
| Buffer needed for Process [mL] | 1000 |
| Bag Size; Cell Collection Bag [mL] | 150 |
| Bag Size: Negative Fraction Bag [mL] | 500 |
| or Non-Target Cell Bag [mL] | |
| Bag Size: Buffer Waste Bag [mL] | 500 |
| Bag Size: Priming Waste Bag [mL] | unchanged |
| or Re-Application Bag [mL] | |
| Additional Process Parameters | |
| Stage length in seconds | 511 |
| Stage length in mL | 85 |

Low-stringency CD34 selection: A second biological sample is enriched by selection for CD34+ cells under low-stringency conditions using a modification of depletion mode use of the Miltenyi Biotec CliniMACS® system as described in Example 1 with modification to intended to provide higher yield of CD34⁺ cells relative to the low-stringency enrichment applied to Patient 1.

Apheresis product from peripheral blood, anticoagulated with Anticoagulant Citrate Dextrose Solution (ACD-A), is provided at either 60×10⁹ to 6×10⁸ total cells (when using one vial of CD34 labeling reagent) or 120×10⁹ to 12×10⁸ total cells (when using two vials of CD34 labeling reagent). In a laminar flow hood under sterile conditions, cell count, cell viability, and count for subpopulations of CD3+, CD19+, or CD34+ cells are measured. The apheresis product is transferred to a transfer bag which is filled to 600 ml total volume with phosphate buffered saline (PBS). The contents are centrifuged at 230 G for 15 minutes and the supernatant is removed, leaving 90 ml to which 5 ml of immunoglobulin is added, followed by mixing at room temperature for 10 minutes. One or two 7.5 ml vials of CliniMACS® CD34 Reagent is injected into the bag, which is mixed and incubated under stirring for 30 minutes at room temperature. The cells are centrifuged at 230 G for 15 minutes and resuspended in in 150 ml of PBS.

The CD34 Selection Program on a CliniMACS® system is run using the sample as described above with the parameters as indicated in Table 2. The process lasts approximately 20-30 minutes. The program is aborted before any of the wash steps take place and the CD34-enriched cells and other non-specific cells are flushed into the “target cell bag”. The “target cell bag” of the CliniMACS® system contains the low-stringency CD34-enriched cell population. The “non-target cell bag” of the CliniMACS® system contains non-target cells.

Low-stringency CD34 selection results in about 35% to 60% yield of CD34⁺ cells and about 10% to 30% relative purity.

Subsequently, both high-stringency CD34-enriched cell population and low-stringency CD34-enriched cell population are each individually transduced or combined and transduced with a recombinant gene therapy vector encoding the FANCA gene product as described in Example 1, and the resulting genetically modified cells are designated as Product 3.1 and Product 3.2, respectively.

If individually transduced product 3.1 and Product 3.2 are mixed together and thereafter administered to Patient 3 by intravenous bolus.

Patient 3 shows rapid in vivo selection engraftment kinetics. This treatment protocol results in multilineage stabilization in hematopoiesis, neutrophils, red blood cells, and platelets. Stabilization of hematopoietic lineages that had declined in Patient 3 prior to transplantation is a convincing endpoint for clinical success.

### Example 4

### Gene Therapy Using Crispr-Cas with High- and Low-Stringency CD34-Enriched Cell Populations

Biological samples are taken from additional patients and high-stringency and low-stringency CD34-enriched cell populations are prepared as described in Example 3. A recombinant gene therapy is created, which is designed to deliver gene editing system capable of directed repair of an endogenous FANC gene. The gene editing system includes a Cas protein or a polynucleotide encoding a Cas protein; a gRNA; and a repair template. The repair template comprises a sequence fragment overlapping known mutation(s) to a FANC gene (e.g. FANCA) in the additional patients. One or both of the high-stringency and low-stringency CD34-enrichment populations are contacted with the recombinant gene therapy vector, and then mixtures of the two CD34-enriched cell populations are autologously transplanted into each of the patients. The patients show rapid in vivo selection engraftment kinetics. This treatment protocol results in multilineage increases in hematopoiesis, neutrophils, red blood cells, and platelets. Recovery of hematopoietic lineages that had declined in the patients prior to transplantation is a convincing endpoint for clinical success.

1. A method for preparing genetically modified cells for the treatment of Fanconi anemia, comprising:
selecting for CD34⁺ cells from a biological sample obtained from a subject by:
(a) applying the biological sample to a capture matrix that binds CD34+ cells;
(b) allowing an unbound fraction of the biological sample to flow through the capture matrix; and
(c) eluting a CD34-enriched cell population from the capture matrix using an elution buffer, wherein the bound fraction is not washed prior to elution,
wherein the CD34-enriched cell population comprises CD34+ cells at a purity of 1% to 45%; and
contacting the CD34-enriched cell population with:
(i) a recombinant gene therapy vector comprising a polynucleotide sequence encoding a Fanconi anemia complementation group (FANC) polypeptide, or a functional variant or fragment thereof; or
(ii) a gene editing system targeting direct repair of an endogenous mutated FANC gene.

2. The method of claim 1, wherein the biological sample is peripheral blood or bone marrow.

3. The method of claim 1, wherein the biological sample is peripheral blood obtained after the subject has been treated with G-CSF, plerixafor, or a combination of G-CSF and plerixafor.

4. The method of claim 1, wherein the CD34-enriched cell population comprises CD34+ cells at a purity of 1% to 30%.

5. The method of claim 1, wherein the selection results in a yield of 35% to 60% of CD34+ cells.

6. The method of claim 1, wherein the selection is performed by bead-based magnetic selection.

7. The method of claim 1, wherein the selection is performed using antibodies or functional fragments thereof that specifically bind to CD34.

8. The method of claim 1, wherein the selection is performed using a flow rate of 10-20 mL/min.

9. The method of claim 1, wherein the CD34-enriched cell population is genetically modified by contacting the CD34-enriched cell population with the recombinant gene therapy vector, wherein the recombinant gene therapy vector comprises a polynucleotide sequence comprising in the following 5′ to 3′ order:
(a) a eukaryotically active promoter sequence; and
(b) a sequence encoding a human FANC polypeptide, or a functional fragment or variant thereof;
wherein the sequence encoding the human FANC polypeptide or functional fragment or variant thereof is operably linked to the eukaryotically active promoter sequence; and wherein the FANC polypeptide is selected from FANCA, FANCC, and FANCG.

10. The method of claim 9, wherein the FANC polypeptide is FANCA.

11. The method of claim 9, wherein the promoter is a human phosphoglycerate kinase 1 (hPGK) promoter.

12. The method of claim 11, wherein the hPGK promoter has at least 90% identity to SEQ ID NO:7, or nucleotides 3541-4051 of SEQ ID NO:24, or nucleotides 1-511 of SEQ ID NO:25.

13. The method of claim 9, wherein the recombinant gene therapy vector comprises a polynucleotide sequence comprising in the following 5′ to 3′ order:
(a) the eukaryotically active promoter sequence;
(b) the sequence encoding the human FANC polypeptide, or functional fragment or variant thereof; and
(c) a mutated woodchuck hepatitis virus post-transcriptional regulatory element (WPRE).

14. The method of claim 13, wherein the mutated WPRE has at least 90% identity to SEQ ID NO:23, or nucleotides 8502-9178 of SEQ ID NO: 24.

15. The method of claim 9, wherein the recombinant gene therapy vector is a lentiviral gene therapy vector.

16. The method of claim 15, wherein the lentiviral gene therapy vector comprises an expression cassette comprising:
a) a 5′ LTR or a modified 5′ LTR;
b) a cPPT sequence;
c) a human phosphoglycerate kinase 1 (hPGK) promoter;
d) a sequence encoding a human FANC protein, or functional fragment or variant thereof,
e) a mutated woodchuck hepatitis virus post-transcriptional regulatory element (WPRE); and
f) a 3′ LTR or a modified 3′ LTR.

17. The method of claim 16, wherein the expression cassette comprises the sequence corresponding to nucleotides 3541 to 9178 of SEQ ID NO: 24.
